(19) United States
(12) Patent Application Publication
(10) Pub. No.: US 2014/0303425 A1
(43) Pub. Date: Oct. 9, 2014

(54) METHOD AND APPARATUS FOR ELECTROMAGNETIC TREATMENT OF COGNITION AND NEUROLOGICAL INJURY

(71) Applicant: IVIVI HEALTH SCIENCES, LLC, San Francisco, CA (US)

(72) Inventors: Arthur A. Pilla, Oakland, NJ (US); Andre' A. Dimino, Woodcliff, NJ (US); Sean Hagberg, San Francisco, CA (US); Steven M. Gluckkstern, San Francisco, CA (US)

(21) Appl. No.: 14/354,587

(22) PCT Filed: Nov. 5, 2012
(86) PCT No.: PCT/US2012/063576

§ 371 Date: Apr. 27, 2014

Related U.S. Application Data
(60) Provisional application No. 61/556,068, filed on Nov. 4, 2011.

(51) Int. Cl. A61N 2/02; A61B 5/11; A61B 5/03; A61B 5/0488; G01R 33/48; A61B 6/03; A61B 5/0476; A61B 5/00; A61N 2/00

(52) U.S. Cl. CPC A61N 2/02; A61B 5/4836; A61B 5/11; A61B 5/031; A61B 5/746; A61B 5/6803; A61N 2/004; G01R 33/4806; A61B 6/037; A61B 5/0476; A61B 5/0488
USPC 600/14; 600/15; 600/13

(57) ABSTRACT

Methods and devices for providing therapeutic electromagnetic field treatment to a subject having a cognitive or neurological condition or injury. Treatment devices can include headwear incorporating electromagnetic treatment delivery devices providing electromagnetic treatment to a user's head area. Such devices include protective headwear such as helmets with electromagnetic delivery devices. Additionally, embodiments of the invention provide for wearable and adjustable electromagnetic treatment devices that can be used to provide electromagnetic treatment to multiple areas of the user's head. Embodiments of the invention provide for sequential electromagnetic treatment with a single or a plurality of treatment applicators which target a single or multiple cerebral regions as determined by imaging, non-imaging and physiological monitoring before, during and after electromagnetic treatment.

### CROSS REFERENCE TO RELATED APPLICATIONS

This application claims priority to U.S. provisional patent application 61/556,068, filed Nov. 4, 2011, and titled “METHOD AND APPARATUS FOR ELECTROMAGNETIC TREATMENT OF COGNITION AND NEUROLOGICAL INJURY”.

This application may also be related to any of the following patent applications, each of which is herein incorporated by reference in its entirety: U.S. patent application Ser. No. 11/003,108, filed Dec. 3, 2004, now U.S. Pat. No. 7,744,524 (“APPARATUS AND METHOD FOR ELECTROMAGNETIC TREATMENT OF PLANT, ANIMAL AND HUMAN TISSUE, ORGANS, CELLS AND MOLECULES”); U.S. patent application Ser. No. 12/771,954, filed Apr. 30, 2010, titled “APPARATUS AND METHOD FOR ELECTROMAGNETIC TREATMENT OF PLANT, ANIMAL AND HUMAN TISSUE, ORGANS, CELLS AND MOLECULES”; U.S. patent application Ser. No. 12/772,002, filed Apr. 30, 2010, titled “APPARATUS AND METHOD FOR ELECTROMAGNETIC TREATMENT OF PLANT, ANIMAL AND HUMAN TISSUE, ORGANS, CELLS AND MOLECULES”; U.S. patent application Ser. No. 12/819,956, filed Jun. 21, 2010, titled “APPARATUS AND METHOD FOR ELECTROMAGNETIC TREATMENT”; U.S. patent application Ser. No. 11/114,666, filed Apr. 26, 2005, now U.S. Pat. 7,740,574, titled “ELECTROMAGNETIC TREATMENT INDUCTION APPARATUS AND METHOD FOR USING SAME”; U.S. patent application Ser. No. 11/223,073, filed Sep. 10, 2005, now U.S. Pat. No. 7,758,490, titled “INTEGRATED COIL APPARATUS FOR THERAPEUTICALLY TREATING HUMAN AND ANIMAL CELLS, TISSUES AND ORGANS WITH ELECTROMAGNETIC FIELDS AND METHOD FOR USING SAME”; U.S. patent application Ser. No. 12/082,944, filed Apr. 14, 2008, now U.S. Pat. No. 7,896,797, titled “ELECTROMAGNETIC FIELD TREATMENT APPARATUS AND METHOD FOR USING SAME”; U.S. patent application Ser. No. 12/819,956, field on Jun. 21, 2010, titled “APPARATUS AND METHOD FOR ELECTROMAGNETIC TREATMENT”; U.S. patent application Ser. No. 13/252,114, filed Oct. 3, 2011, titled “METHOD AND APPARATUS FOR ELECTROMAGNETIC TREATMENT OF HEAD, CEREBRAL AND NEURAL INJURY IN ANIMALS AND HUMANS”; and U.S. patent application Ser. No. 13/285,761, filed Oct. 31, 2011, and titled “METHOD AND APPARATUS FOR ELECTROMAGNETIC ENHANCEMENT OF BIOCHEMICAL SIGNALING PATHWAYS FOR THERAPEUTICS AND PROPHYLAXIS IN PLANTS, ANIMALS AND HUMANS.”

### INCORPORATION BY REFERENCE

All publications and patent applications mentioned in this specification are herein incorporated by reference to the same extent as if each individual publication or patent application was specifically and individually indicated to be incorporated by reference.

### FIELD

Described herein are electromagnetic treatment devices, systems and methods. Some embodiments pertain generally to a method and apparatus for therapeutic and prophylactic treatment of animal and human nervous system. For example, some embodiments described are devices, systems and methods for delivering electromagnetic signals and fields to individuals at risk of suffering neurological injuries. In particular, headgear such as helmets having electromagnetic treatment delivery device that can be activated by sensors are described. Additionally, some embodiments described provide for delivering electromagnetic signals and fields to individuals suffering from a neurological injury. Specifically, embodiments provide designs such as multi-coil applicator configured to provide therapeutic electromagnetic field treatment to a single or combinations of multiple regions of a user's head as the therapy requires. Additionally, some embodiments described provide for delivering electromagnetic signals and fields to individuals who may benefit from enhanced cognitive responses beneficial in training or task learning. Specifically, embodiments provide designs such as applicators with a plurality of applicators placed in appropriate head gear which may be programmed to provide electromagnetic field treatment to a single cerebral region or combinations of multiple regions of a user's head in the sequence required by the task or training involved.

Other embodiments pertain to use of non-thermal time-varying electromagnetic fields configured to accelerate the asymmetrical kinetics of the binding of intracellular ions to their respective binding proteins which regulate the biochemical signaling pathways living systems employ to contain and reduce the inflammatory response to injury. Other embodiments pertain to the non-thermal application of repetitive pulse bursts of sinusoidal, rectangular, chaotic or arbitrary waveform electromagnetic fields to instantaneously accelerate ion-buffer binding in signaling pathways in animal and human nervous system using ultra lightweight portable coupling devices such as inductors and electrodes, driven by miniature signal generator circuitry.

Another embodiment pertains to application of sinusoidal, rectangular, chaotic or arbitrary waveform electromagnetic signals, having frequency components below about 100 GHz, configured to accelerate the binding of intracellular calcium (Ca²⁺) to a buffer, such as calmodulin (CaM), to enhance biochemical signaling pathways in animal and human nervous systems. Signals configured according to some embodiments produce a net increase in a bound ion, such as Ca²⁺ at CaM binding sites because the asymmetrical kinetics of Ca/CaM binding allows such signals to accumulate voltage induced at the ion binding site, thereby accelerating voltage-dependent ion binding. Examples of therapeutic and prophylactic applications of the present invention are modulation of biochemical signaling in anti-inflammatory pathways, modulation of biochemical signaling in cytokine release pathways, modulation of biochemical signaling in growth factor release pathways; up regulation or down regulation of any messenger ribonucleic acid (mRNA), or gene, associated with the release of any cytokine, growth factor or protein modulated by EMF; edema and lymph reduction, anti-inflammatory, post-surgical and post-operative pain and edema relief, nerve, bone and organ pain relief, increased local blood flow, microvascular blood perfusion, treatment of tissue and organ ischemia, brain tissue ischemia from stroke or traumatic brain injury, treatment of neurological injury and neurodegenerative diseases such as Alzheimer's and Parkinson's, or any other cognitive or motor impairment; angiogenesis, neovascularization; enhanced immune response; enhanced effectiveness of pharmacological agents; nerve regeneration; prevention of apoptosis; modulation of heat shock proteins for prophylaxis and response to injury or pathology.

Some embodiments can also be used in conjunction with other therapeutic, diagnostic and prophylactic procedures and modalities such as MRI, fMRI, PET, SPECT, EEG, EMG and any other cognitive measure, and heat, cold, light, ultrasound, mechanical manipulation, massage, physical therapy, wound dressings, orthopedic and other surgical fixation devices, and surgical interventions. In addition, any of the variations described herein can also be used in conjunction with one or more pharmacological agents. Any of the variations described herein can also be used with any other imaging or non-imaging diagnostic procedures.

In some variations the systems, devices and/or methods generally relate to application of electromagnetic fields (EMF), and in particular, pulsed electromagnetic fields (PEMF), including a subset of PEMF in a radio frequency domain (e.g., pulse-modulated radio frequency or PRF), for the treatment of head, cerebral and neural injury, including neurodegenerative conditions in animals and humans, as well as to improve cognitive abilities in normal subjects or to treat or prevent cognitive impairment in subjects with cognitive disorders.

### BACKGROUND

Over the past 40 years, it has been found that the application of weak non-thermal electromagnetic fields (“EMF”) can result in physiologically meaningful in vivo and in vitro bioeffects. Time-varying electromagnetic fields, comprising PEMF or PRF, ranging from several Hertz to about 100 GHz , have been found to be clinically beneficial when used as a therapy for reducing pain levels for patients undergoing surgical procedures, promoting healing in patients with chronic wounds or bone fractures, and reducing inflammation or edema in injuries (e.g. sprains).

Although PEMF/PRF therapy has been used for a variety of treatments, one challenge has been in providing a PEMF/PRF delivery device in a design configuration that accommodates the patient's injury and concurrent treatment. For example, EMF devices are difficult to use with patients who are bed-ridden, bandaged, and engaged in ongoing treatment (or monitoring) by metal-containing devices. Some embodiments of present invention provide for configurations of EMF delivery devices that can accommodate such situations where access to the injured area is limited.

In addition to the access challenge discussed above, there is also a need to provide EMF treatment to patients close in time to a neurological injury. Immediate or substantially immediate medical treatment can greatly reduce the damage that arises from a head injury. Some embodiments described provide for protective articles such as helmets that initiate EMF treatment once a threshold event occurs. Contemplated embodiments include helmets with incorporated EMF devices that activate once a sensor measures an impact of sufficient value.

Beginning in the 1960's, development of modern therapeutic and prophylactic devices was stimulated by clinical problems associated with non-union and delayed union bone fractures. Early work showed that an electrokinetic pathway could be a means through which bone adaptively responds to mechanical input. Early therapeutic devices used implanted and semi-invasive electrodes delivering direct current (“DC”) to a fracture site. Non-invasive technologies were subsequently developed using electric and electromagnetic fields. These modalities were originally created to provide a non-invasive means of inducing an electrical/mechanical waveform at a cell/tissue level. Clinical applications of these technologies in orthopaedics have led to approved applications by regulatory bodies worldwide for treatment of bone repair in non-union and fresh fractures, as well as spine fusion.

Presently several EMF devices constitute the standard armamentarium of orthopaedic clinical practice for treatment of difficult to heal fractures. The success rate for these devices has been very high. The database for this indication is large enough to enable its recommended use as a safe, non-surgical, non-invasive alternative to a first bone graft. Additional clinical indications for these technologies have been reported in double blind studies for treatment of avascular necrosis, tendinitis, osteoarthritis, wound repair, blood circulation, pain from arthritis and other musculoskeletal pathologies, and post-operative pain and edema.

In addition, cellular studies have addressed the effects of weak electromagnetic fields on both signal transduction pathways and growth factor and cytokine regulation. It has been shown that EMF instantly modulates CaM-dependent nitric oxide (NO) signaling, which, in turn, modulates cyclic guanosine monophosphate (cGMP), which, in turn modulates the up- or down-regulation of the genes involved in the production of the growth factors and cytokines necessary for tissue repair and growth. Ion/ligand binding at intracellular buffers are generally considered an initial EMF target pathway structure. The clinical relevance to treatments, for example, of bone repair, is up-regulation such as modulation, of growth factor and cytokine production as part of normal molecular regulation of bone repair. Cellular level studies have shown effects on CaM-dependent signaling, calcium ion transport, cell proliferation, the up- and down-regulation of Interleukin-1beta (IL-1β), Insulin Growth Factor (“IGF-II”) , and IGF-II receptor expression in osteoblasts. Effects on Insulin Growth Factor-I (“IGF-I”) and IGF-II have also been demonstrated in rat fracture callus. Further studies demonstrated an increase in both TGF-β mRNA and protein in osteoblast cultures resulting from a direct effect of EMF on a CaM-dependent pathway. Cartilage cell studies have shown similar increases in TGF-β1 mRNA and protein synthesis from EMF, demonstrating a therapeutic application to joint repair. Cellular studies have also demonstrated that the EMF enhancement of NO and cGMP release can be blocked by CaM antagonists such as N-(6-Aminohexyl)-5-chloro-1-naphthalenesulfonamide hydrochloride (W-7) and trifluoroperazine (TFP), showing that CaM-dependent NO signaling is involved in tissue repair and growth.

It is also well known that CaM-dependent nitric oxide (NO) signaling modulates nervous system activity. In particular, NO signaling plays a significant role in the rhythmic slow activity in the hippocampus that affects learning and cognition in general. Furthermore, NO signaling modulates the neuronal differentiation that is involved in plasticity. Therefore, since EMF signals can modulate CaM-dependent NO signaling, it is believed that EMF signals can be configured to affect nervous system growth, maintenance and activity.

It is further believed that EMF signals can be configured to modulate the ionic-dependent signalings that govern the biochemical pathways organisms employ for tissue growth, repair and maintenance. It is further believed that EMF signals can be configured to modulate calcium ion (Ca²⁺)-dependent CaM signaling pathways which modulate tissue repair and maintenance, and reduce inflammation, pain, and edema. In particular, EMF signals can be used to accelerate the binding of Ca²⁺ to CaM. As Ca²⁺ ions bind to CaM, it undergoes a conformational change after which CaM can bind to and activate a number of key enzymes involved in cell viability and function, such as the endothelial and neuronal constitutive nitric oxide synthases (cNOS); eNOS and nNOS, respectively. Activation of these enzymes results in a transient production of NO, which is anti-inflammatory. In contrast, the persistent increases in NO produced by inducible NOS, (iNOS), which is not Ca²⁺ dependent, are pro-inflammatory. CaM-dependent NO activates soluble guanylyl cyclase (sGC), which catalyzes the formation of cyclic guanosine monophosphate (cGMP). The CaM/NO/cGMP signaling pathway can rapidly modulate blood flow in response to normal physiologic demands, as well as to inflammation. This same pathway can modulate the up- or down-regulation of growth factors such as basic fibroblast growth factor (FGF-2) and vascular endothelial growth factor (VEGF), as well as the up- or down-regulation of cytokines such as Interleukin-1beta (IL-1β), resulting in pleiotropic effects on cells involved in tissue repair and maintenance. EMF may also up regulate or down regulate the messenger ribonucleic acid (mRNA), or gene, associated with particular proteins involved in tissue repair and maintenance (e.g., growth factor or cytokine).

While the primary and immediate consequences of mechanical trauma to neurons cannot be undone, secondary pathological sequelae, specifically brain swelling and inflammation, are situational candidates for intervention. The toll of neurological deficits and mortality from TBI continue in the military and private sectors and, to date, there are no widely successful medical or surgical interventions to prevent neuronal death. Current medical practice has attempted to use pharmaceuticals to mitigate and prevent tissue damage and injury resulting from secondary physiological responses of traumatic brain injury with little success. For example, intravenous, high-dose corticosteroids have been administered to reduce cerebral inflammation after traumatic brain injury, but several studies have demonstrated that steroids can be neurotoxic. In fact, results from a clinical randomized trial in 2005 tested whether a high dose regimen of the steroid methylprednisolone sodium succinate (MPSS), administered within 8 hours after injury, would improve survival after head injury. This trial was planned to randomize 20,000 patients and was powered to detect a drop in mortality from 15% to 13%, a small, but important improvement in outcome. However, the data and safety monitoring board halted the trial after half of the patients were enrolled as it became apparent that MPSS significantly increased mortality of severe injuries from 17.9% to 21.1% (P=0.0001).

Given the paucity of treatment options for head trauma, cognitive disorders, and cognitive improvement, there is a need for a therapy that can non-invasively target the brain or regions of the brain to modulate neurotransmitter release for cognitive outcomes or to reduce secondary physiological responses such as inflammation, swelling, and intracranial pressure while also promoting repair and regrowth in and around the injured area.

While EMF treatments have been explored for a variety of uses, the possible benefits of EMF in treating or preventing neurological injury and degenerative conditions such as traumatic brain injury (TBI), subarachnoid hemorrhage, brain ischemia, stroke, and Alzheimer's or Parkinson's Disease are relatively unknown. This is in part due to the fact that the inflammatory response in the central nervous system (CNS) differs somewhat from that of the periphery systems for which EMF signals are currently used. In comparison, for example, inflammation and swelling in the CNS can lead to secondary tissue damage and neuronal death. Moderate to severe TBI can produce mechanical damage characterized by the disruption of cell membranes and blood vessels, resulting in direct and ischemic neuronal death. Moreover, inflammation and swelling reduces blood flow to the brain and can cause damage and death of healthy brain tissue. Even in the absence of direct mechanical injury (i.e. diffuse brain trauma), astrocytes and microglia react to these conditions and will secrete cytokines (e.g. IL-1β, TNF-α, IFN-γ, and IL-6) and as well as other pro-inflammatory molecules, such as glutamate, reactive oxygen and nitrogen species, and it is well-known that these factors, alone, and in combination, can be neurotoxic.

Because neurological injury such as head trauma can induce a cascade of molecular, cellular, and vascular responses to produce brain inflammation and swelling, which can then lead to secondary injury or death, there is a need for a therapy that can quickly and specifically target injured neuronal cells and neuronal biochemical pathways to reduce inflammation and promote tissue repair and regrowth. However, a significant challenge has been that current available EMF devices are difficult to use with patients who are bed-ridden, heavily bandaged, and/or wearing surgical, monitoring, or metal containing devices that can interfere with the delivery of therapeutic EMF. For example, a TBI patient may be placed in an immobilizing body support article such as a head and neck brace during transport to a hospital, which limits access by EMF devices to the injured region. Some embodiments of the present invention provide for various configurations of EMF delivery devices that can accommodate such situations where access to the injured area is limited. Moreover, some embodiments of the present invention can be incorporated into an anatomical positioning device such as a dressing, bandage, compression bandage, compression dressing; head, neck or other body portion wraps and supports; garments; furniture; and other body supports to provide EMF treatment directly. In further embodiments, the methods and devices contemplated may include a sensor that monitors a patient's condition such that if a change occurs, the delivery device may modify the treatment automatically to accommodate the change.

In addition to the above, there is also a need to provide EMF treatment to patients as soon as possible after injury where medical attention is not immediate. After sustaining an injurious event such as a fall, patients are often left minimally assisted or completely unassisted for minutes to several hours. Because every moment following a neurological injury matters in preventing death or additional injury, there is a need to provide EMF treatment immediately after injury. As such, some aspects of the present invention can be incorporated into protective articles such as headgear (helmets) which will provide EMF treatment once a threshold event has occurred. For example, one embodiment contemplated provides for a football helmet or a military helmet with an EMF device that activates once the device registers an impact of sufficient force.

Further to the above, because many of the same pathways affected by neurological injury are also at issue in neurodegenerative disorders and conditions (e.g. inflammation of brain tissue in Alzheimer's Disease, or cognitive impairment), some embodiments of the present invention may provide for treatment of neurological disorders with the EMF devices and treatments described.

Treatment for improving cognition has been limited to the use of pharmaceuticals (e.g. psychostimulants or cholinergic agents) that can target neurotransmitters or neuropathways in the central nervous system (CNS). For example, attention has been given to acetylcholinesterase inhibitors such as tacrine that can inhibit the breakdown of the neurotransmitter acetylcholine. However, reliance on pharmaceutical treatments has several drawbacks including limited bioavailability of the drug and severe adverse side effects such as vomiting, convulsions, and bradycardia. Furthermore, once administered, it is often difficult to completely limit the pharmacokinetics and effects of a psychopharmaceutical to a single target neuropathway. For example, typical antipsychotic drugs (e.g. haloperidol) that target the brain's dopamine pathways have the unwanted side effect of blocking other dopamine pathways, which can cause extrapyramidal motor side effects that can persist long after the medication is discontinued.

To avoid the severe and often dangerous drawbacks of pharmaceutical treatment, some embodiments provide for methods and devices using noninvasive EMF to treat a subject affected by cognitive impairment or disorder. It is believed that applying EMF to regions of the brain will improve the subject's ability to execute cognitive processes such as a learning, memory-processing, perception, and problem solving by, for example, enhancing appropriate neurotransmitter release, or by improving plasticity by enhancing the differentiation of in situ neurons.

Further embodiments provide for methods and devices using noninvasive EMF to improve cognitive function in subjects suffering from a cerebral or neuronal injury. Some embodiments are directed to providing treatment to TBI patients in need of relearning basic tasks such as language and bodily functions affected by the injury.

In addition to providing noninvasive devices and methods for treating cognitive impairment patients suffering from injury, disorders, or disease (e.g. Alzheimer's and dyscalculia), other embodiments provide for methods and devices for improving cognitive abilities in a normal subject not suffering from cognitive impairment. This need is especially apparent for military personnel who must be quickly trained or retrained in the use of new military technology, equipment, and systems, for which they may have had little or no exposure to prior to their military service. Moreover, in combat situations, it is critical for service men and women to be functioning at the highest level of cognition possible to avoid fatal mishaps.

Furthermore, some embodiments provide for methods and devices for improving cognitive abilities where the methods and devices are applied while the subject is engaged in an activity and the subject's performance of that activity improves during or after application of the treatment/device. In these embodiments, the device may be configured for ease of use while the subject is engaged in the activity. For example, in a combat situation, methods and devices contemplated herein may be used to improve the subject's surveillance and target acquisition abilities while the surveillance or acquisition is ongoing. In such circumstances, the EMF methods or device may be configured to provide treatment in a convenient manner that does not interfere with the subject's duties (e.g. treatment through a combat helmet).

In addition, the devices and methods described can also be used to help non-military individuals quickly learn new skills and information. For example, the methods and devices described can be used to help children or adults to quickly learn new skills or information for educational or career development.

Additional embodiments can improve specific cognitive functions by providing treatments to areas of the brain known or shown to be active when a subject is engaged in a particular task such as calculation or learning. In some embodiments, a subject's brain activity may be mapped while the subject is engaged in an activity to determine the target areas for treatment.

To facilitate the use of the methods and devices described, some embodiments of the present invention can be incorporated into furniture or articles of clothing such as hats, headbands, helmets etc. to provide EMF treatment.

Moreover, an embodiment according to the present invention can also be used in conjunction with other therapeutic and prophylactic procedures and modalities such as heat, cold, light, ultrasound, mechanical manipulation, massage, physical therapy, wound dressings, orthopedic and other surgical fixation devices, and surgical interventions.

### SUMMARY OF THE DISCLOSURE

Some embodiments described herein are devices, systems and methods for delivering electromagnetic signals and fields to individuals at risk of suffering neurological injuries. Some embodiments described provide for protective headgear such as helmets that incorporate an electromagnetic field treatment device. The helmets (or other headgear) may include a sensor configured to measure a parameter of the environment, helmet, or the user such as impact or trauma force. The sensor can also be configured to trigger activation of the treatment device and delivery of the electromagnetic field to the user. The sensor may be prompt activation of the treatment device once the sensor measures a sensed value that satisfies or exceeds a predetermined threshold value.

Some embodiments provide for a protective helmet apparatus for delivering electromagnetic treatment comprising a helmet shell having an opening adapted to receive the head of a user, at least a layer of padding within the helmet shell configured to provide comfort and reduce impact forces on the head of the user, an electromagnetic treatment device at least partially within the helmet shell, and a sensor coupled to helmet, the sensor configured to detect an impact parameter and to activate the electromagnetic treatment device when the impact parameter exceeds a predetermined threshold.

Some embodiments provide for headgear designed to incorporate a plurality coils positioned to apply EMF to a single cerebral region or to a combination of cerebral regions to enhance cognition or to enhance learning and administered in combination with imaging, non-imaging and electrophysiological diagnostic modalities.

Optionally, in any of the preceding embodiments, the electromagnetic treatment device includes an applicator configured to deliver a therapeutic electromagnetic field to the user's head and a control circuit controlling a generator configured to provide an electromagnetic signal to the applicator to induce the therapeutic electromagnetic field with a sequence and regimen appropriate to the therapeutic need.

Optionally, in any of the preceding embodiments, the electromagnetic signal can comprise a carrier signal having a frequency in a range of about 0.01 Hz to about 10,000 MHz and a burst duration from about 0.01 to about 1000 msec.

Optionally, in any of the preceding embodiments, the sensor is an accelerometer and/or a pressure sensor.

Optionally, in any of the preceding embodiments, the sensor is configured to monitor the impact parameter while the helmet is worn by the user and to activate the electromagnetic treatment device once a measured impact parameter exceeds a threshold value.

Optionally, in any of the preceding embodiments, the electromagnetic treatment device is configured to apply a pre-programmed treatment protocol.

Optionally, in any of the preceding embodiments, the headgear or helmet includes an alert means for indicating that the electromagnetic treatment device is active.

Optionally, in any of the preceding embodiments, the sensor measures an impact force and/or a shockwave force experienced by the user.

Optionally, in any of the preceding embodiments, the electromagnetic treatment device is removable from the headwear or helmet. In other embodiments, the electromagnetic treatment device is incorporated into the headwear or helmet.

Optionally, in any of the preceding embodiments, the electromagnetic treatment device is configured to generate the electromagnetic signal through an electrode separated from a target tissue location by an air gap.

Optionally, in any of the preceding embodiments, the applicator is configured to contact the user's scalp.

Optionally, in any of the preceding embodiments, the electromagnetic treatment device comprises a replaceable or rechargeable power source.

Optionally, in any of the preceding embodiments, a remote control element is included and configured to operate the electromagnetic treatment device.

Optionally, in any of the preceding embodiments, the applicator comprises pliable and conformable coils having a generally circular shape.

Optionally, in any of the preceding embodiments, the applicator has a diameter between about 2 inches to about 8 inches.

Optionally, in any of the preceding embodiments, the applicator is adjustable.

Optionally, in any of the preceding embodiments, the applicator comprises a flexible band configured to electrically and physically couple to the circuit control generator.

Optionally, in any of the preceding embodiments, the applicator comprises a collapsible wire having a retracted and extended position.

Optionally, in any of the preceding embodiments, the applicator is removably attached to the headwear or helmet with a fastening mechanism.

Optionally, in any of the preceding embodiments, the applicator comprises conductive ink.

Optionally, in any of the preceding embodiments, a connecting member is included between the applicator and the control circuit. Optionally, in any of the preceding embodiments, a connecting member comprises a pliable material adapted to allow the applicator and the control circuit to move relative to each other.

Optionally, in any of the preceding embodiments, a processor is included and configured to collect and record user information while the apparatus is worn.

Optionally, in any of the preceding embodiments, the electromagnetic device is configured to emit a pulse-modulated radio frequency signal with a carrier frequency of approximately at 27.12 MHz at a 2 msec burst repeating at about 2 bursts/sec. Optionally, in any of the preceding embodiments, the electromagnetic signal comprises a carrier signal below 1 MHz. In some embodiments, the electromagnetic signal generated by the control circuit and generator has a carrier frequency within the ISM band. Optionally in any of the preceeding embodiments the electromagnetic signal comprises symmetrical or asymmetrical pulses having a pulse duration between about 0.1 and about 10,000 μsec, with a burst duration between about 100 and 10,000 μsec, and a repetition rate between 0.1 and 100 Hz. Optionally, in any of the preceding embodiments, the electromagnetic treatment device comprises a set of interchangeable applicators, the set of interchangeable applicators configured to be attachable and removable from the headwear or helmet independent from the circuit control generator.

Optionally, in any of the preceding embodiments, the applicator comprises a flexible printed circuit board.

Other embodiments described provide for devices, systems, and methods for delivering electromagnetic signals and fields to individuals suffering from neurological injuries. Such embodiments include a delivery device having an applicator with a plurality or multiple coils capable of delivering an electromagnetic field to a target region. The multi-coil applicator may be made from a metal containing material such as a metal wire. Additionally, the coils of the applicator may be connected to one another by way of a connecting member that is configured to calibrate the frequency of an electromagnetic signal received by the applicator. The connecting member may also connect the multi-coil applicator to a lead or connector that attaches to a power source and/or signal generator.

Some described embodiments provide for an electromagnetic treatment delivery device having a multi-coil applicator configured to apply a therapeutic electromagnetic field to multiple locations on a user's head, wherein the multi-coil applicator comprises a plurality of non-concentric conductive coils. The delivery device may include a control circuit configured to control a generator, wherein the generator is coupled to the multi-coil applicator and configured to provide a pulse-modulated radio frequency signal to the multi-coil applicator to induce the therapeutic electromagnetic field.

Optionally, in any of the preceding embodiments, the electromagnetic treatment delivery device may include a connecting member connecting the plurality of conductive coils to each other and to the generator.

Optionally, in any of the preceding embodiments, the electromagnetic treatment delivery device may include an article of headwear configured to be worn by a user, wherein the multi-coil applicator is incorporated into the headwear.

Optionally, in any of the preceding embodiments, the multi-coil applicator forms a figure eight pattern.

Optionally, in any of the preceding embodiments, the multi-coil applicator comprises pliable and conformable coils having generally circular shapes.

Optionally, in any of the preceding embodiments, at least two coils of the multi-coil applicator each have a diameter between about 6 inches to about 8 inches.

Optionally, in any of the preceding embodiments, the multi-coil applicator is configured to generate an electric field on at least two hemispheres of the user's head.

Optionally, in any of the preceding embodiments, the delivery device is incorporated into a bandage.

Optionally, in any of the preceding embodiments, the delivery device includes a sensor configured to monitor a user parameter. Optionally, in any of the preceding embodiments, the user parameter monitored is intracranial pressure.

Optionally, in any of the preceding embodiments, the control circuit is configured to control the device to deliver a pre-programmed treatment protocol.

Described herein are also devices, systems and methods for delivering electromagnetic signals and fields configured specifically to accelerate the asymmetrical kinetics of the binding of intracellular ions to their respective intracellular buffers, to enhance the biochemical signaling pathways animals and humans employ to respond to nervous system injury from stroke, traumatic brain injury, head injury, cerebral injury, neurological injury, neurodegenerative diseases and cognitive impairment.

One variation according to the present invention utilizes repetitive arbitrary non-thermal EMF waveforms configured to maximize the bound concentration of intracellular ions at their associated molecular buffers to enhance the biochemical signaling pathways living systems employ in response to nervous system injury from stroke, traumatic brain injury, head injury, cerebral injury, neurological injury, neurodegenerative diseases and cognitive impairment. Non-thermal electromagnetic waveforms are selected first by choosing the ion and the intracellular binding protein, for example Ca²⁺ and CaM, among the many ion-buffer combinations within the living cell, which determines the frequency range within which the signal must have non-thermal frequency components of sufficient, but non-destructive, amplitude to accelerate the kinetics of ion binding. Signals comprise a pulse duration, random signal duration or carrier period which is less than half of the ion bound time to increase the voltage in the target pathway so as to maximally accelerate ion binding to maximally modulate biochemical signaling pathways to enhance specific cellular and tissue responses to nervous system injury from stroke, traumatic brain injury, head injury, cerebral injury, neurological injury, neurodegenerative diseases and cognitive impairment.

In some variations, signals comprise bursts of at least one of sinusoidal, rectangular, chaotic or random EMF wave shapes; have burst duration less than about 100 msec, with frequency content less than about 100 MHz, repeating at less than about 1000 bursts per second. Peak signal amplitude in the ion-buffer binding pathway is less than about 1000 V/m. Another embodiment comprises about a 1 to about a 50 millisecond burst of radio frequency sinusoidal waves in the range of about 1 to about 100 MHz, incorporating radio frequencies in the industrial, scientific and medical (hereinafter known as ISM) band, for example 27.12 MHz, but it may be 6.78 MHz, 13.56 MHz or 40.68 MHz in the short wave frequency band, repeating between about 0.1 and about 100 bursts/sec. Such waveforms can be delivered via inductive coupling with a coil applicator or via capacitive coupling with electrodes in electrochemical contact with the conductive outer surface of the target.

Some embodiments described provide for a waveform configuration that accelerates the kinetics of Ca²⁺ binding to CaM, consisting of about a 1 to about a 10 msec burst of between about 5 MHz to about 50 MHz including frequencies in the ISM band, repeating between about 1 and about 5 bursts/sec and inducing a peak electric field between about 1 and about 100 V/m, then coupling the configured waveform using a generating device such as ultra lightweight wire or printed circuit coils that are powered by a waveform configuration device such as miniaturized electronic circuitry.

Other embodiments described provide for a waveform configuration that accelerates the kinetics of Ca²⁺ binding to CaM, consisting of about a 1 to about a 10 msec burst of 27.12 MHz radio frequency sinusoidal waves, repeating between about 1 and about 5 bursts/sec and inducing a peak electric field between about 1 and about 100 V/m, then coupling the configured waveform using a generating device such as ultra lightweight wire, printed circuit coils or conductive garments that are powered by a waveform configuration device such as miniaturized electronic circuitry which is programmed to apply the aforementioned waveform at fixed or variable intervals, for example for 1 minute every 10 minutes, or for 10 minutes every hour, or for any other regimen found to be beneficial for a prescribed treatment. Further embodiments provide for methods and devices for applying electromagnetic waveforms to animals and humans that accelerate the asymmetrical kinetics of the binding of intracellular ions to their associated intracellular buffers, by configuring the waveforms to contain repetitive and/or non-repetitive frequency components of sufficient amplitude to maximize the bound concentration of the intracellular ion to its associated intracellular buffer, thereby to enhance the biochemical signaling pathways living tissue employ in response to nervous system injury from stroke, traumatic brain injury, head injury, cerebral injury, neurological injury, neurodegenerative diseases and cognitive impairment.

Additional embodiments provide for methods and devices for applying electromagnetic waveforms to animals and humans which accommodate the asymmetrical kinetics of the binding of Ca²⁺ to CaM by configuring the waveforms to contain repetitive and/or non-repetitive frequency components of sufficient amplitude to accelerate and increase the binding of Ca²⁺ to CaM, thereby enhancing the CaM-dependent nitric oxide (NO)/cyclic guanosine monophosphate (cGMP) signaling pathway.

Further embodiments provide for electromagnetic waveform configurations to contain repetitive and/or non-repetitive frequency components of sufficient amplitude to accelerate and increase the binding of Ca²⁺ to CaM, thereby enhancing the CaM-dependent NO/cGMP signaling pathway to accelerate blood and lymph vessel dilation for relief of post-operative and post traumatic pain and edema.

Another aspect of the present invention is to configure electromagnetic waveforms to contain repetitive and/or non-repetitive frequency components of sufficient amplitude to accelerate and increase the binding of Ca²⁺ to CaM, thereby enhancing the CaM-dependent NO/cGMP signaling pathway, or any other signaling pathway, to enhance angiogenesis and microvascularization for nervous system repair.

A further aspect of the present invention is to configure electromagnetic waveforms to contain repetitive and/or non-repetitive frequency components of sufficient amplitude to accelerate and increase the binding of Ca²⁺ to CaM, thereby enhancing the CaM-dependent NO/cGMP signaling pathway, or any other signaling pathway, to accelerate deoxyribonucleic acid (hereinafter known as DNA) synthesis by living cells.

Another aspect of the present invention is to configure electromagnetic waveforms to contain repetitive and/or non-repetitive frequency components of sufficient amplitude to accelerate and increase the binding of Ca²⁺ to CaM, thereby enhancing the CaM-dependent NO/cGMP signaling pathway to up- or down-regulate specific genes (messenger ribonucleic acid, mRNA) which control growth factor release, such as basic fibroblast growth factor (bFGF), vascular endothelial growth factor (VGEF), bone morphogenic protein (BMP), or any other growth factor production by living cells.

Another aspect of the present invention is to configure electromagnetic waveforms to contain repetitive and/or non-repetitive frequency components of sufficient amplitude to accelerate and increase the binding of Ca²⁺ to CaM, thereby enhancing the CaM-dependent NO/cGMP signaling pathway to modulate growth factor release, such as basic fibroblast growth factor (bFGF), vascular endothelial growth factor (VGEF), bone morphogenic protein (BMP), or any other growth factor production by living cells.

It is yet another aspect of the present invention to configure electromagnetic waveforms to contain repetitive and/or non-repetitive frequency components of sufficient amplitude to accelerate and increase the binding of Ca²⁺ to CaM, thereby enhancing the CaM-dependent NO/cGMP signaling pathway to up regulate or down regulate specific genes (mRNA) which modulate growth factor and cytokine release, such as basic fibroblast growth factor (bFGF), vascular endothelial growth factor (VGEF), bone morphogenic protein (BMP), IL-1β, or any other growth factor or cytokine production living cells employ in response to nervous system injury from stroke, traumatic brain injury, head injury, cerebral injury, neurological injury, neurodegenerative diseases and cognitive impairment.

Another aspect of the present invention is to configure electromagnetic waveforms to contain repetitive and/or non-repetitive frequency components of sufficient amplitude to accelerate and increase the binding of Ca²⁺ to CaM, thereby enhancing the CaM-dependent NO/cGMP signaling pathway, or any other signaling pathway, to modulate cytokine, such as interleukin 1-beta (IL-1β), interleukin-6 (IL-6), or any other cytokine production by living cells, as well as to up regulate or down regulate the associated gene(s) (mRNA).

Another aspect of the present invention is to configure electromagnetic waveforms to contain repetitive and/or non-repetitive frequency components of sufficient amplitude to accelerate and increase the binding of Ca²⁺ to CaM, thereby enhancing the CaM-dependent NO/cGMP signaling pathway, or any other signaling pathway, to modulate cytokine, such as interleukin 1-beta (IL-1β), interleukin-6 (IL-6), or any other cytokine production by living cells in response to nervous system injury from stroke, traumatic brain injury, head injury, cerebral injury, neurological injury, neurodegenerative diseases and cognitive impairment.

Another aspect of the present invention is to configure electromagnetic waveforms to contain repetitive and/or non-repetitive frequency components of sufficient amplitude to accelerate and increase the binding of Ca²⁺ to CaM, thereby enhancing the CaM-dependent NO/cGMP signaling pathway, or any other signaling pathway, to accelerate or decelerate the production of intra- and extra-cellular proteins by up regulating or down regulating the appropriate gene(s) (mRNA) for tissue repair and maintenance.

It is another aspect of the present invention to configure electromagnetic waveforms to contain repetitive and/or non-repetitive frequency components of sufficient amplitude to accelerate and increase the binding of Ca²⁺ to CaM, thereby enhancing the CaM-dependent NO/cyclic adenosine monophosphate (cAMP) signaling pathway, or any other signaling pathway, to modulate cell and tissue differentiation.

It is yet another aspect of the present invention to configure electromagnetic waveforms to contain repetitive and/or non-repetitive frequency components of sufficient amplitude to accelerate and increase the binding of Ca²⁺ to CaM, thereby enhancing the CaM-dependent NO/cAMP signaling pathway, or any other signaling pathway, to prevent or reverse neurodegeneration.

It is yet another aspect of the present invention to configure electromagnetic waveforms to contain repetitive and/or non-repetitive frequency components of sufficient amplitude to accelerate and increase the binding of Ca²⁺ to CaM, thereby modulating the CaM-dependent NO/cAMP signaling pathway, or any other signaling pathway, to modulate the neurotransmitter releases involved in cognition.

Another aspect of the present invention is to configure electromagnetic waveforms to contain frequency components of sufficient amplitude to accelerate the binding of Ca²⁺ to CaM, thereby enhancing the CaM-dependent NO/cGMP signaling pathway to modulate heat shock protein release from living cells.

Other embodiments provide for methods and devices to improve neuronal survival.

The above and yet other embodiments and advantages of the present invention will become apparent from the hereinafter set forth Brief Description of the Drawings and Detailed Description of the Invention.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings of which:

FIG. 1 is a flow diagram of a method for treating a neurological condition/injury, including cognitive impairment, according to an embodiment of the devices and methods described herein.

FIG. 2 illustrates a device for application of electromagnetic signals according to an embodiment of the devices and methods described herein.

FIG. 3 illustrates placement of a device for application of electromagnetic signals according to an embodiment of the devices and methods described on a posterior region of the head.

FIG. 4A illustrates an apparatus for application of electromagnetic signals according to an embodiment.

FIG. 4B illustrates an apparatus for application of electromagnetic signals according to an embodiment with multiple applicators and control circuit/signal generators.

FIG. 5 illustrates placement of a device for application of electromagnetic signals according to an embodiment of the devices and methods described in proximity to a lateral cerebellar hemisphere.

FIG. 6 illustrates placement of a device for application of electromagnetic signals to an anterior region of the head.

FIG. 7 illustrates an electromagnetic treatment apparatus integrated into a head and face support garment according to an embodiment of the devices and methods described.

FIG. 8 illustrates an electromagnetic treatment apparatus integrated into an alternative head and face support garment according to an embodiment of the devices and methods described.

FIG. 9 illustrates placement of a device for application of electromagnetic signals to a region of a canine head.

FIG. 10 illustrates an electromagnetic treatment apparatus integrated into bedding material according to some embodiments.

FIGS. 11A-D illustrate an electromagnetic treatment apparatus integrated into headgear according to some embodiments.

FIGS. 12A-B illustrate an electromagnetic treatment apparatus integrated into alternative headgear according to some embodiments.

FIGS. 13A-13B illustrate the placement of an electromagnetic treatment apparatus in headgear according to some embodiments.

FIG. 14 illustrates an insert for headgear.

FIGS. 15A-B illustrates an electromagnetic treatment apparatus having multiple applicator/generating members integrated into headgear.

FIGS. 16A-E illustrate an apparatus for application of electromagnetic signals according to an embodiment having an elastic band.

FIG. 17 illustrates the effect of an EMF signal configured according to embodiments described on nitric oxide (NO) release from MN9D neuronal cell cultures.

FIG. 18 illustrates the effect of an EMF signal configured according to embodiments described on cyclic adenosine monophosphate (cAMP) release from MN9D neuronal cell cultures.

FIG. 19 compares the effect of an EMF signal configured according to embodiments described and exogenous cAMP on neurite outgrowth from MN9D neuronal cell cultures.

FIG. 20 illustrates an electromagnetic treatment apparatus integrated into a hat.

FIGS. 21A-21D illustrate a figure eight design for an electromagnetic treatment apparatus.

FIGS. 22A-22B illustrate a low frequency electromagnetic treatment apparatus.

FIG. 23 illustrates a signal generator that can be connected to applicator/generating members of an electromagnetic treatment delivery device.

FIG. 24 illustrates an alternative signal generator that can be connected to applicator/generating members of an electromagnetic treatment delivery device.

FIG. 25 is a block-diagram of a PEMF treatment and cognition system according to described embodiments.

FIG. 26 shows a training session protocol.

### DETAILED DESCRIPTION

Some embodiments described herein are devices, systems and methods for delivering electromagnetic signals and fields to individuals at risk of suffering neurological injuries. In particular, embodiments described provide for protective headgear such as helmets that include electromagnetic treatment devices incorporated into the helmet. The helmets (or other headgear) may include a sensor configured to measure a parameter of the environment, helmet, or the user such as impact or trauma force. In some cases, the sensor senses the impact force experienced by the wearer. If the sensed impact force (such as shockwave force) reaches a predetermined threshold value, the electromagnetic treatment device is designed to activate and apply treatment. This allows treatment of a potentially life-threatening neurological injury to begin almost immediately or shortly after a threshold event such as an explosion.

In addition, other embodiments described herein are devices, systems, and methods for delivering electromagnetic signals and fields to individuals suffering from neurological injuries. A significant problem with providing electromagnetic treatment to such patients has been delivering electromagnetic field treatment while accommodating the patient's existing medical treatment, which usually includes bed-rest, bandages, and medical equipment containing metal. In order to accommodate these treatments, some embodiments described provide for a multi-coil applicator electromagnetic delivery device. The delivery device includes a multi-coil applicator that is designed to provide treatment to different regions of the user's head without interfering with existing treatment. In some cases, the delivery device includes a two coil applicator forming a figure eight design that applies an electric field to two different regions of the user's head. The two coil applicator design can be incorporated into bandages. Moreover, the delivery device can be designed to minimize additional hardware needed near the target treatment region. The two coils may be connected by a single connecting member that connects to a power source and/or signal generator. Additional details regarding the embodiments described above will be provided in a later section.

By way of background, it is believed that induced time-varying electric fields using capacitively or inductively coupled EMF may be configured to affect neurological tissue including specific cellular/molecular pathways in CNS or peripheral tissues allowing these tissues to react in a physiologically meaningful manner. For example, a waveform may be configured within a prescribed set of parameters so that a particular pathway, such as CaM-dependent NO synthesis within the neurological tissue target, is modulated specifically.

In other embodiments, PEMF applied prior to, during and after a traumatic event may provide protection from or reduction in injury, for example, through the activation of heat inducible factor-1 (HIF-1), through induction of heat shock proteins, including heat shock protein (HSP) 70 and/or through the expression of neuroglobin and/or cytoglobin. In some embodiments, the PEMF modulates through the calcium/calmodulin pathway, which, in turn, can increase the expression of calcium/calmodulin dependent protein kinases, including CaM PK II. This can then also increase HIF-1 expression, which then induces the expression of HSP 70, as well as cytoglobin.

Both the applied waveform and the dosing or treatment regime applied may be configured so that at least this pathway is targeted specifically and effectively. Furthermore, the stimulation protocol and dosing regimen may be configured so that an electromagnetic signal applicator device may be portable/wearable, lightweight, require low power, and does not interfere with medical or body support such as wound dressings, orthopedic and other surgical fixation devices, and surgical interventions.

In some embodiments, a method of treating a subject for a neurological condition or disease includes applying the one or more (or a range of) waveforms that are needed to target the appropriate pathways in the target neuronal tissue. This determination may be made through calculation of mathematical models such as those described in U.S. Pat. Nos. 7,744,524, 7,740,574 and U.S. Patent Publication Nos. 2011-0112352 filed Jun. 21, 2010 as U.S. patent application Ser. No. 12/819,956 and 2012-0089201 filed as U.S. patent application Ser. No. 13/285,761 (herein incorporated by reference) to determine the dosing regimen appropriate for a modulating a molecular pathway (e.g. Ca/CaM pathway).

For example, it is believed that pathways involved in the maintenance and repair of cerebral tissue include the Ca/CaM pathway. To modulate this pathway, in some variations, the electromagnetic signals applied are configured to comprise bursts of at least one of sinusoidal, rectangular, chaotic or random wave shapes; burst duration less than about 100 msec, with frequency content less than about 100 GHz at 1 to 100,000 bursts per second. In other variations, the electromagnetic signals have about a 1 msec to about a 50 msec burst of radio frequency sinusoidal waves in the range of about 1 to about 100 MHz, incorporating radio frequencies in the industrial, scientific, and medical band, for example 27.12 MHz, 6.78 MHz, or 40.68 MHz, repeating between about 0.1 to about 10 bursts/sec. The carrier signal frequency may also lie within the ranges commonly utilized for wireless communication devices such as about 800 MHz, about 2000 MHz and about 7000 MHz. Alternatively, the carrier signal frequency may be below 1 MHz, such as 100 Hz or 1 Hz. In such variations, the lower carrier signal frequency may require a longer burst duration, e.g. 30 msec at an amplitude of between about 0.001 G and 1 G. In further variations an EMF signal can be applied that consists of a 2 msec burst of 27.12 MHz sinusoidal waves repeating at 2 bursts/sec.

Electromagnetic signals can be applied manually or automatically through application devices to provide a range of treatment ranges and doses. For example, PEMF signals can be applied for 15 minutes, 30 minutes, 60 minutes, etc. as needed for treatment. Electromagnetic signals can also be applied for repeated durations such as for 15 minutes every 2 hours. The electromagnetic applicator devices can also provide a time varying magnetic field (for example, peak=0.05 G, Average=10⁻³ G) to induce a time varying electric field (for example average=30V/m) in the tissue target. Moreover, each signal burst envelope may be a random function providing a means to accommodate different electromagnetic characteristics of target tissue. Similarly, the number of treatments and the dose regime may be varied depending on the progress of the target location.

In some embodiments, modifying neuronal pathways can result in increased or decreased cerebral blood flow to a target location. For example, modulating the Ca/CaM pathway can cause vasodilation in the target cerebral tissue. Vasodilation of cerebral tissue can result in increased cerebral blood flow which can mitigate inflammation, neuronal degeneration, and tissue death and promote tissue regrowth, repair, and maintenance.

As is understood by one of ordinary skill in the art, the terms neurological condition, disease, injury etc. as used herein are not intended to be limited to any particular condition or injury described. A neurological injury can mean at least an injury that results from mechanical damage arising from an initial insult or trauma event and any secondary injury from secondary physiological responses. In some embodiments, the methods and devices contemplated may be configured to treat patients for whom the trauma event is initiated by medical personnel as part of another treatment. For example, in the case of a craniotomy to remove brain tumors or lesions, the neurological injury would include the surgical incision(s) into brain tissue and subsequent secondary injury from resulting inflammation or swelling that develops after the initial insult. Similarly, neurological conditions or diseases can mean at least, and non-exhaustively, degenerative disorders such as Alzheimer's or neurological, functional, or behavioral impairment(s) resulting from injury. For example, secondary physiological responses such as inflammation can damage healthy brain tissue which can result in impairment of a cognitive or behavioral function associated with that part of the brain.

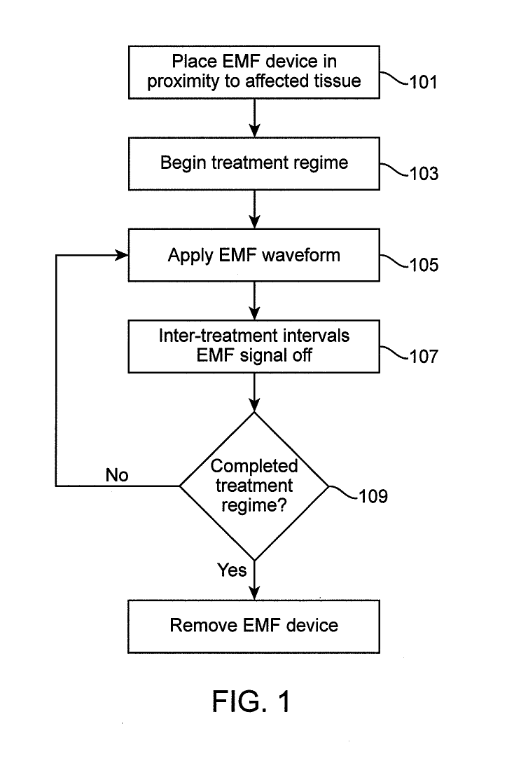

FIG. 1 is a flow diagram of a method for treating a subject with a neurological condition, such as cognitive impairment, or injury. In some variations, before beginning the treatment, one or more (or a range of) waveforms may be determined that target the appropriate pathway for the target tissue. In other variations, one or more (or a range of) waveforms may be determined that target the appropriate region of the brain. The region targeted by the electromagnetic field may differ depending on the cognitive ability at issue. For example, studies have shown that the hippocampus is likely involved in processing memory and spatial navigation. To improve memory retention or retrieval, the PEMF treatment may be directed toward the temporal lobe of the brain in close proximity to the hippocampus. Alternatively, if learning speech or language is the cognitive activity at issue, Broca's area may be the target location for treatment. Similarly, to improve problem solving skills, the frontal lobe may be the general target treatment location.

As can be appreciated, any number or combinations of target locations may be treated as needed. Because how the brain processes and develops can be extremely complex and individualized, a subject may undergo a mapping or imaging procedure, such as positron emission tomography (PET), magnetoencephalography (MEG), or magnetic resonance imaging (MRI), to determine the target area(s) to be treated. Additionally, once the active target area(s) of the brain are determined for particular cognitive tasks, treatment can be applied to target areas to specifically improve function in that area.

Once the treatment parameter and/or target area is determined, electromagnetic signals are applied to the target location. As described in FIG. 1, a method of treating a subject with a neurological injury or condition (or for improving cognition) may include the step of placing the tissue to be treated (e.g. near one or more CNS regions) in contact, or in proximity to, an EMF device 101. Any appropriate EMF device may be used. In general, the device may include an applicator (e.g. inductor applicator) which may be placed adjacent to or in contact with the target location/tissue. The device may also contain a signal conditioner/processor for forming the appropriate waveform to selectively and specifically modulate a pathway (e.g. Ca/CaM pathway). In further embodiments, the device may include a timing element (e.g. circuit) for controlling the timing automatically after the start of the treatment.

In the example shown in FIG. 1, once treatment begins 103, the device, in some variations, applies EMF (e.g. pulse-modulated high-frequency) waveforms at low amplitude (e.g. less than 1 milliGauss, less than 10 milliGauss, less than 50 milliGauss, less than 100 milliGaus, less than 200 milliGauss, etc.) The EMF (e.g. pulse-modulated high-frequency) waveform can then be repeated at a particular frequency after an appropriate delay. This repetitive waveform can be repeated for a first treatment time (e.g. 5 minutes, 15 minutes, 20 minutes, 30 minutes, etc.) and then followed by a delay during which the treatment is “off” 107. This waiting interval or inter-treatment treatment interval may last for minutes or hours (15 minutes, 2 hours, 4 hours, 8 hours, 12 hours, etc.) and then the treatment interval may be repeated again until the treatment regime is complete 109. Once treatment is completed, the EMF device can be removed from contact or proximity to the patient.

In some variations, the treatment device is pre-programmed (or configured to receive pre-programming) to execute the entire treatment regime (including multiple on-periods and/or intra-treatment intervals) punctuated by predetermined off-periods (inter-treatment intervals) when no treatment is applied. In further variations, the device is pre-programmed to emit a pulse-modulated radio frequency signal at 27.12 MHz consisting of a 2 msec burst repeating at 2 bursts/sec.

In other embodiments, the treatment may be provided while the subject is engaged in a skill or activity that can be affected by improved cognitive abilities. For example, the subject may be engaged in learning how to solve mathematical problems when the treatment regime begins 103. The subject can continue to engage in the activity while the device applies PEMF 105. Similarly, the subject may continue the activity during the inter-treatment interval or after the treatment is completed. Advantageously, in some variations, the skill or activity learning process is unaffected by the treatment regime and the subject does not need to discontinue the activity in order to receive treatment. This is particularly beneficial where it is necessary to quickly train the subject in a new skill and further delay for separate cognitive treatment is not ideal.

In some variations, the cognitive improvement treatment and new activity/skill may be engaged in alternating steps. The subject may first provide baseline data set indicating her cognitive abilities for a specific activity prior to treatment. Then the subject may be treated with a first iteration of PEMF at certain treatment parameters. Following the first treatment, the subject may be tasked with performing the new activity or skill to provide a comparison data set. In the event that the comparison data set and the baseline data set indicate the improvement in cognitive abilities is not sufficient, the treatment parameters may be adjusted (e.g. modify waveform, frequency, burst duration, target location etc.). This treatment modification and adjustment step may be repeated until a set of treatment parameters is determined that will provide acceptable improvement. Once the treatment parameters are determined, the subject may engage in further treatment, which can be done either during or separately from engaging in the activity or skill.

In some embodiments, data sets may be collected by utilizing brain imaging techniques such as MRI, PET, or MEG etc. A set of pre-treatment data may be taken and compared to a post-treatment data set. Data may be collected during or separately from the performance of tasks, activities, or skills. In further embodiments, the electromagnetic field delivery device may be pre-programmed to run through a range of treatment parameters while the subject is engaged in a cognitive activity and collect or access data regarding the subject's performance of the activity during that treatment. For example, the delivery device may communicate directly with measuring devices or indirectly through an interface such as a computer or processor. In such cases, the delivery device may run through a range of treatment parameters and collect data for each set of parameters. For example, the device applies treatment parameters A and collects data set A′. Then the device may pause for an inter-treatment interval before apply treatment parameters B to collect data set B′. The device may run through a number of treatment parameters to collect a range of data sets for the different treatment parameters. Once the data sets are collected, the device may determine (e.g. through a processor) which treatment parameter is suitable for the subject and continue with treatment at those parameters.

As can be appreciated, the described treatment and devices for improving cognition can be used to treat healthy subjects or subjects suffering from neurological conditions or injuries. In the latter case, subjects suffering from neurological conditions or injuries such as TBI often experience diminished cognitive skills as a result of the injury. In such cases, some embodiments provide treatments to help subjects relearn or improve cognitive skills such as language, memory, or bodily functions. Additionally, the use of cognitive function, cognition, cognitive skills etc. as used herein is not meant to limit these phrases to any particular set of cognitive abilities. Rather, the phrases broadly refer to all brain processes involved in mental and physical tasks such as memory retention/enhancement, calculation, hand-eye coordination, etc.

In further embodiments, the delivery device provides dynamic treatment options where the treatment parameters may be modified during treatment according to the subject's response. For example, the device may include feedback sensors configured to monitor the subject's physiological responses to the applied electromagnetic fields. In some cases, the subject device may shut off automatically if the sensors indicate a monitored condition is outside an acceptable range. In other embodiments, the device may notify treatment staff that a position adjustment is needed where the subject is accessing a different portion of the brain for the cognitive activity.

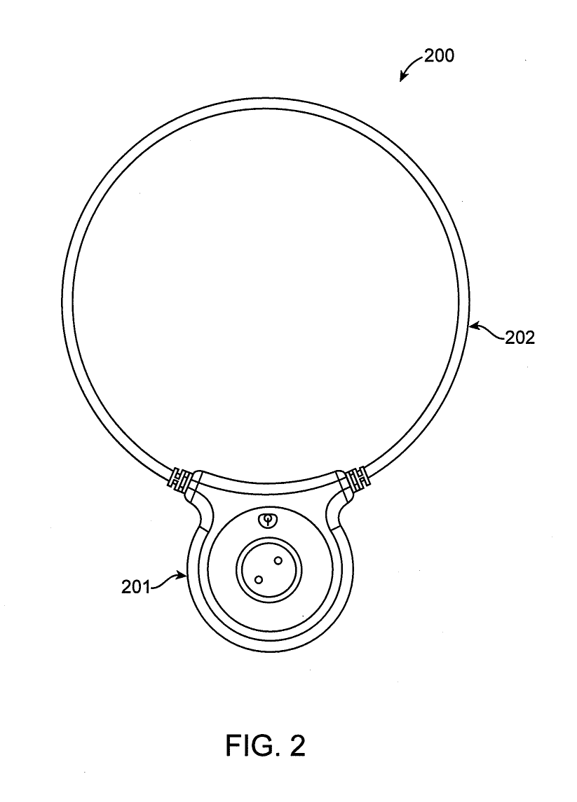

FIG. 2 illustrates an embodiment of an apparatus 200 that may be used. The apparatus is constructed to be self-contained, lightweight, and portable. A control circuit/signal generator 201 may be held within a (optionally wearable) housing and connected to a applicator/generating member such as an electrical coil 202. In some embodiments, the control circuit/signal generator 201 is constructed in a manner that given a target pathway within a target tissue, it is possible to choose waveform parameters that satisfy a frequency response of the target pathway within the target tissue. For some embodiments, control circuit/signal generator 201 applies mathematical models or results of such models that describe the dielectric properties of the kinetics of ion binding in biochemical pathways.

In further embodiments, the device 200 may include a processing component for collecting, accessing, or assessing data regarding the subject's condition (e.g. cognitive abilities or intracranial pressure) before, during, and after treatment. The processing component may be present within the control circuit 201 or anywhere else suitable on device 200. In variations, the processing component may be separate from the device 200; however, the processing component may communicate with the device 200 to provide data regarding the treatment.

Waveforms configured by the control circuit/signal generator 201 are directed to a generating member/applicator 202. In some variations, the generating member/applicator 202 comprises electrical coils that are pliable and comfortable. In further embodiments, the generating member/applicator 202 is made from one or more turns of electrically conducting wire in a generally circular or oval shape, any other suitable shape. In further variations, the electrical coil is a circular wire applicator with a diameter that allows encircling of a subject's cranium. In some embodiments, the diameter is between approximately 6-8 inches. In general, the size of the coil may be fixed or adjustable and the control circuit/signal generator may be matched to the material and the size of the applicator to provide the desired treatment.

The apparatus 200 may deliver a pulsing magnetic field that can be used to provide treatment of a neurological condition or injury. In some embodiments, the device 200 may apply a pulsing magnetic field for a prescribed time and can automatically repeat applying the pulsing magnetic field for as many applications as are needed in a given time period, e.g. 6-12 times a day. The device 200 can be configured to apply pulsing magnetic fields for any time repetition sequence. When electrical coils are used as a generating member/applicator 202, the electrical coils can be powered with a time varying magnetic field that induces a time varying electric field in a target tissue location.

In other embodiments, an electromagnetic signal generated by the generating member/applicator 202 can be applied using electrochemical or capacitive coupling, wherein electrodes are in direct contact with skin or another outer electrically conductive boundary of the target tissue (e.g. skull or scalp). In other variations, the electromagnetic signal generated by the generating member/applicator 202 can also be applied using electrostatic coupling wherein an air gap exists between a generating member/applicator 202 such as an electrode and the target tissue. In further examples, a signal generator and battery is housed in the miniature control circuit/signal generator 201 and the miniature control circuit/signal generator 201 may contain an on/off switch and light indicator. In other variations, the power source (e.g. battery) can be replaced or is rechargeable.

In further embodiments, the activation and control of the treatment device may be done via remote control such as by way of a fob that may be programmed to interact with a specific individual device. In other variations, the treatment device further includes a history feature that records the treatment parameters carried out by the device such that the information is recorded in the device itself and/or can be transmitted to another device such as computer, smart phone, printer, or other medical equipment/device.

In other variations, the treatment device 200 has adjustable dimensions to accommodate fit to a variety of patient head sizes. For example, the generating member/applicator 202 may comprise modular components which can be added or removed by mated attaching members. Alternatively, the treatment device 200 may contain a detachable generating member/applicator (e.g. detachable circular coil or other configurations) that can be removed and replaced with configurations that are better suited for the particular patient's needs. A circular coil generating member/applicator 202 may be removed and replaced with an elongate generating member/applicator such that EMF treatment can be applied where other medical equipment may obstruct access by a circular generating member/applicator 202. In other variations, the generating member/applicator may be made from Litz wire that allows the generating member/applicator to more easily conform to accommodate different target areas or sizes.

Although shown as an electrical coil in FIG. 2, it is understood that a generating member/applicator of any shape or material may be used if configured to provide the appropriate treatment parameters. For example, in some embodiments, the generating member/applicator includes a series or an array (or arrays) of generating members/applicators rather than a single electrical coil. In such embodiments, the series or array of generating members/applicators can be of any shape suitable for treatment. In some variations, a series of coils may be placed in any combination or orientation relative to one another. The coils may be of the same or differing size and be placed at a range of distances from one another.

In other embodiments, the diameter of a circular generating member/applicator may be selected based on the volume of the tissue target. In some variations, the depth of penetration for the electromagnetic field increases with increased diameter. In such embodiments, a larger diameter will provide a field of sufficient amplitude within a greater volume allowing for deeper penetration in the target location. Accordingly, by modifying the diameter or size of the generating member/applicator, the depth of the treatment field can be adjusted as needed. Greater depth of penetration may be advantageous where the injured target region is below the surface of the target location. Alternatively, where a greater depth of penetration is not needed, generating members/applicators of smaller size may be more appropriate where surface application is desired. For example, for treatment of a large surface area, an array of smaller sized generating members/applicators can be used to cover a large area without deep penetration beyond the surface.

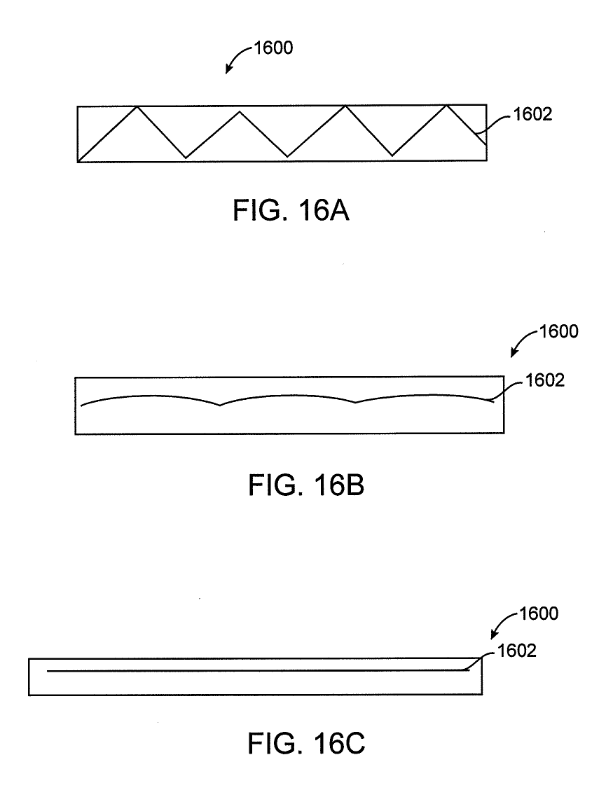

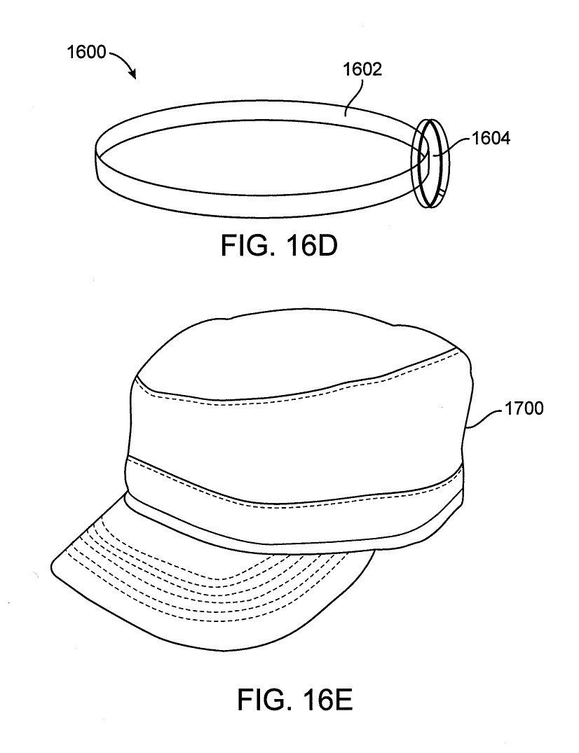

In further embodiments, an adjustable generating member/applicator may include an elastic or flexible band that is configured to electrically and/or physically connect to a signal generator. The elastic or flexible band may include a collapsible wire/coil configured to generate or conduct the waveform transmitted by the signal generator and provide an electromagnetic field to a target location. In some embodiments, the elastic or flexible band is adjustable in size to accommodate a range of head sizes. In other variations, the flexible band may include a locking mechanism for adjusting the band size for a specific subject's head size. For example, the band may include connectors such as slots and hooks (e.g. like a belt) spaced at various lengths so that only a portion of the band length encircles the target location. In further variations, the band may include a collapsible wire that is in a retracted position when unused that can expand to an extended position when placed on a target location. As shown in FIG. 16A, an elastic band 1600 includes a collapsible wire 1602. FIG. 16A shows the collapsible wire 1602 in a retracted position and FIGS. 16B-C show collapsible wire 1602 at different degrees of extension. The flexible band may be connected to the signal generator by way of a connecting member as described above.

In further embodiments, as shown in FIG. 16D, the flexible band may have an embedded applicator and power supply. The embedded applicator may be a wire (optionally collapsible) 1602 that is integrated with the band material and connected physically or electrically to a power supply 1604. In some embodiments, the power supply may not be placed on the flexible band itself. For example, the power supply 1604 may be placed in a pocket and connected by a connecting member to the applicator 1602. In further variations, the flexible band 1600 can be placed in a hat, such as a military cap 1700 (see FIG. 16E). In such cases, the flexible band 1600 may be removably attached to the cap such that the flexible band 1600 may be worn by itself (e.g. headband) or worn as a part of another article such as a hat or helmet. Removably attaching the flexible band to a wearable article may be done by any number of mechanisms known in the art such as Velcro or fabric loops in wearable article for holding the flexible band in place.

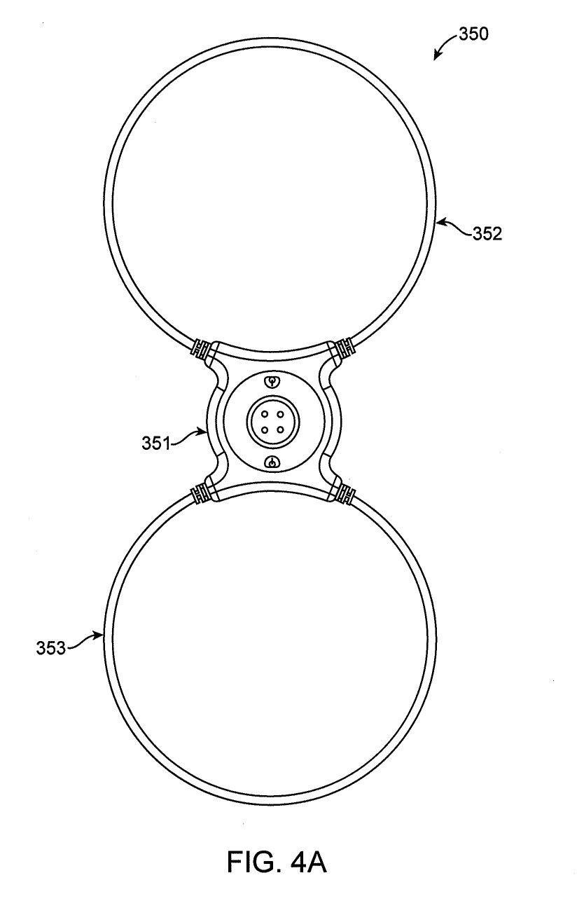

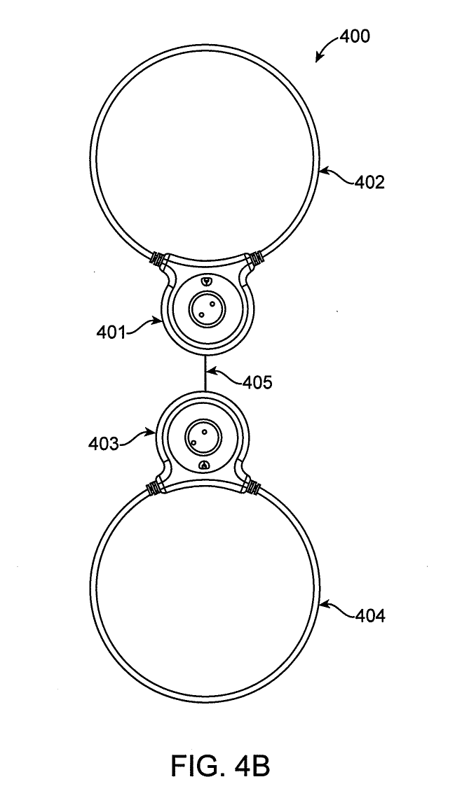

In further embodiments, the generating member/applicator and the control circuit/signal generator may be further separated by a connecting member (see FIG. 4B, connecting member 405) that can provide a physical or electrical connection between the generating member/applicator and the control circuit/signal generator. In addition, the connecting member may be adjustable to provide greater distance between the generating member/applicator and the control circuit/signal generator in order to minimize the proximity between the injured area and the control circuit/signal generator. In further variations, the connecting member may be made from the same or different material than the generating member/applicator. In some embodiments, the connecting member is made from a pliable material that allows the generating member/applicator and control circuit/signal generator to move relative to one another (e.g. bend or twist).

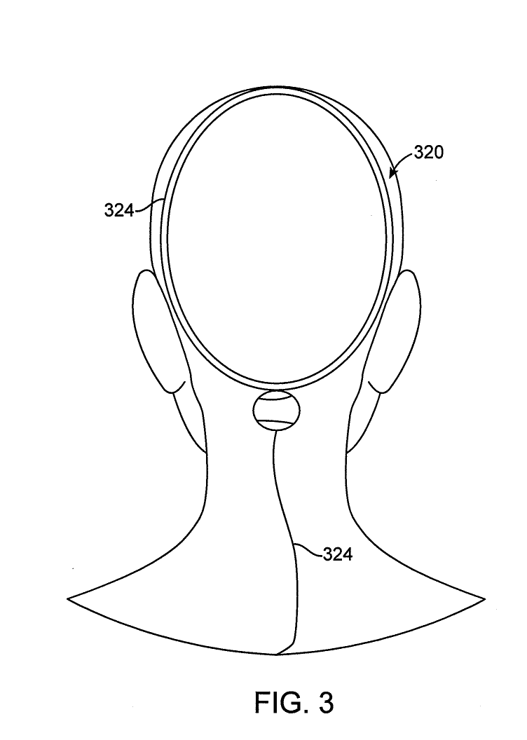

In further embodiments, the EMF method may include a plurality of EMF delivery devices that are positioned in contact or in proximity to various target locations. For example, one device as described in FIG. 2 may be placed on a left hemisphere of a subject's cranium, while another device may be placed on a right hemisphere of a subject's cranium. Similarly, a plurality of devices may be positioned in a variety of regions (e.g. top, bottom, partial rear, temporal lobe, etc.) as needed for treatment. For example, FIG. 3 illustrates an EMF device positioned at the posterior region of the subject's cranium. Furthermore, in other variations, the devices may employ different or same treatment parameters that are operated in staggered or simultaneous combination.

In some embodiments, the generating member/applicator is in close proximity to the target location and the signal generator/control circuit is not placed near the target location. For example, as shown in FIG. 3, the delivery device 320 has connecting member 324 that connects the generating member/applicator 322 to the signal generator (not shown). The signal generator may be placed at a location away from the target treatment region such as attached to a hip belt or in a pocket so that the signal generator does not need to be near the head area.

In further variations, the EMF apparatus may include more than one coil in the generating member/applicator. For example, FIG. 4A illustrates a treatment device 350 with a single miniature control circuit/signal generator 351 with two opposing circular coils 352, 353 for the applicator/generating member. In such an embodiment, the device can employ a figure eight configuration. The device can be placed on the lateral aspect of both cranial hemispheres. As shown, the single control circuit can be configured to control the applicator by providing an electromagnetic signal simultaneously to both coils.

In other embodiments, application of the EMF can be done in alternating or simultaneous cycles. For example, in some treatments, both coils 353, 352 can provide pulsing magnetic fields of the same treatment regime (same frequency, same repetition, etc.) in sync while, in other embodiments, the coils alternate in providing EMF to their respective locations. In some embodiments, one coil may provide an “on” interval while another coil is in an “off” cycle for the same interval and then in a subsequent interval the coils switch on and off positions.

Moreover, some variations may include multiple control circuit/signal generators or more than two generating members/applicators. As shown in FIG. 4B, treatment device 400 includes two control circuit/signal generators 401, 403, two generating members/applicators 402, 404, and connecting member 405. Control circuit/signal generator 401 is configured to transmit EMF waveforms to generating member/applicator 402. Similarly, control circuit/signal generator 403 is configured to transmit EMF waveforms to generating member/applicator 404. In some embodiments, treatment device 400 is configured such that both control circuit/signal generators 401, 403 transmit waveforms simultaneously. In other embodiments, the control circuit/signal generators alternate transmission. In further variations, each control circuit/signal generator is pre-programmed to provide EMF treatment independently of the other control circuit/signal generator. As can be appreciated, any number or combination of treatment parameters may be employed with such EMF devices as needed for a particular patient.

As shown in FIG. 4B, connecting member 405 provides a physical and/or electrical connection between the two control circuit/signal generators 401, 403. In one variation, the connecting member 405 is disposed between a control circuit/signal generator and a generating member/applicator and, in other embodiments, the connecting member may be between two or more generating members/applicators. Furthermore, some variations may contain one or more connecting members where each connecting member is adjustable to allow variability in the dimensions of the treatment device to better accommodate the target treatment location.

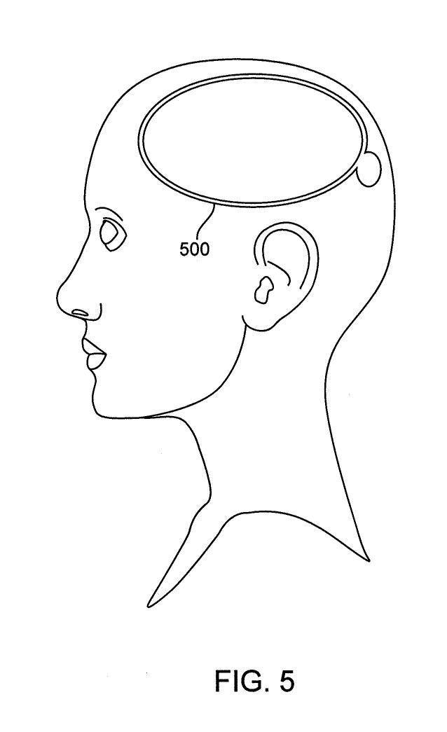

In some embodiments, the devices described herein can be positioned to treat a subject with a traumatic brain injury (and/or in need of improved cognition). As shown in FIG. 5, the EMF device 500 is placed in close proximity to the left cerebellar hemisphere. In further variations, the EMF device may include a figure eight configuration such as those described in FIGS. 4A and 4B, where each lateral hemisphere is in close proximity to a generating member/applicator. In some embodiments, the generating member/applicator is in a figure eight configuration. The figure eight configuration may include a plurality of generating members/applicators or, alternatively, a multi-coil applicator. In some embodiments, the generating member/applicator is connected to a connecting member that connects the generating members/applicators to a control circuit and/or a power source. The power source may be a battery source.

In some embodiments, the applicator or applicators is a coil applicator that can be made from a metal component. The metal component may be flexible, light weight wire. Alternatively, the metal component can be made from a relatively rigid metal material. In other embodiments, the applicator may include conductive materials such as conductive inks placed on a substrate such as fabric.

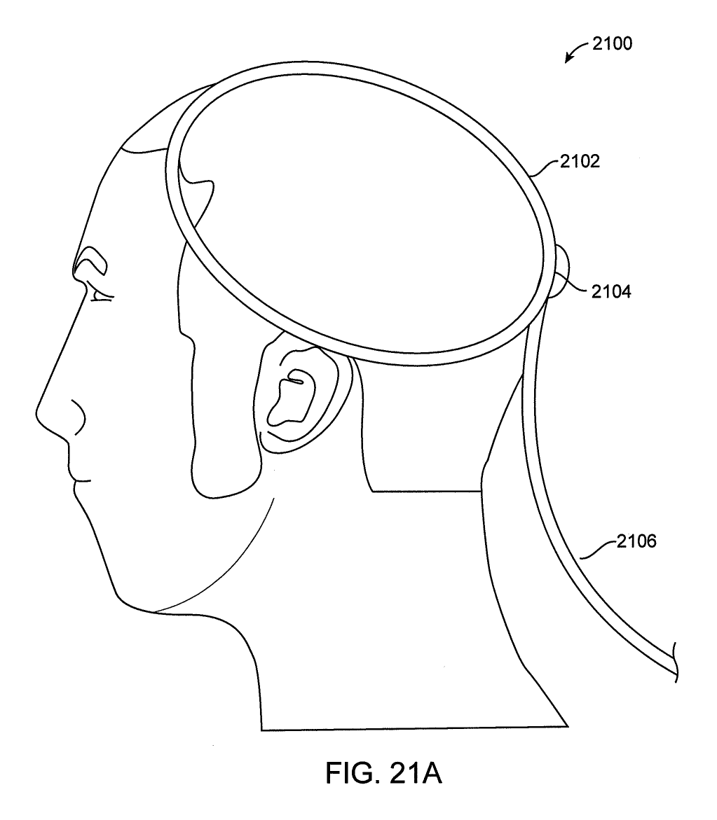

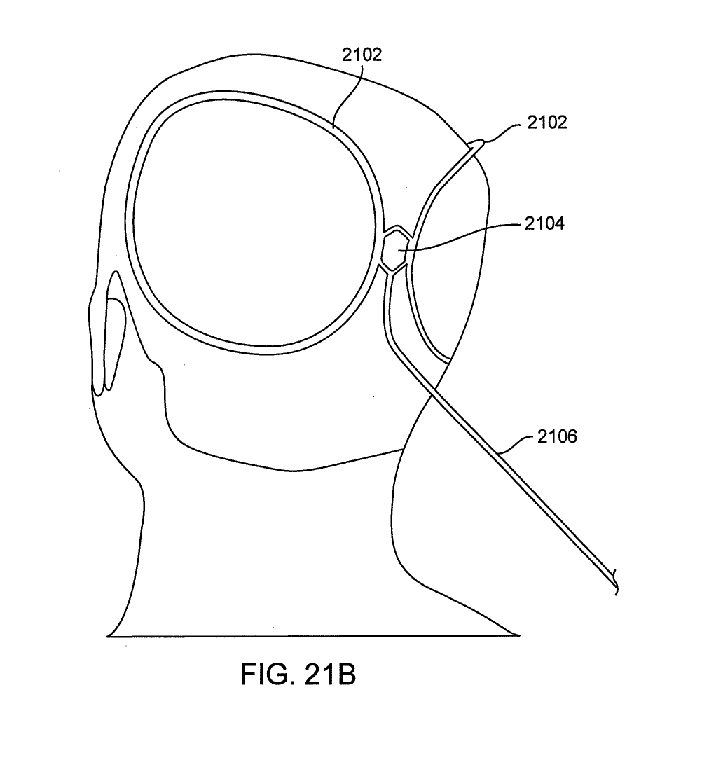

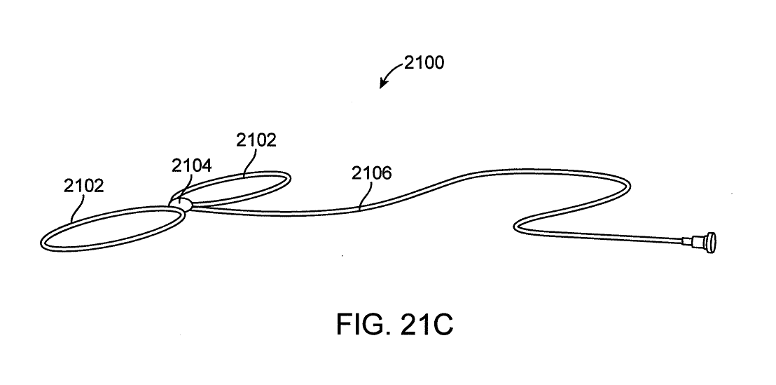

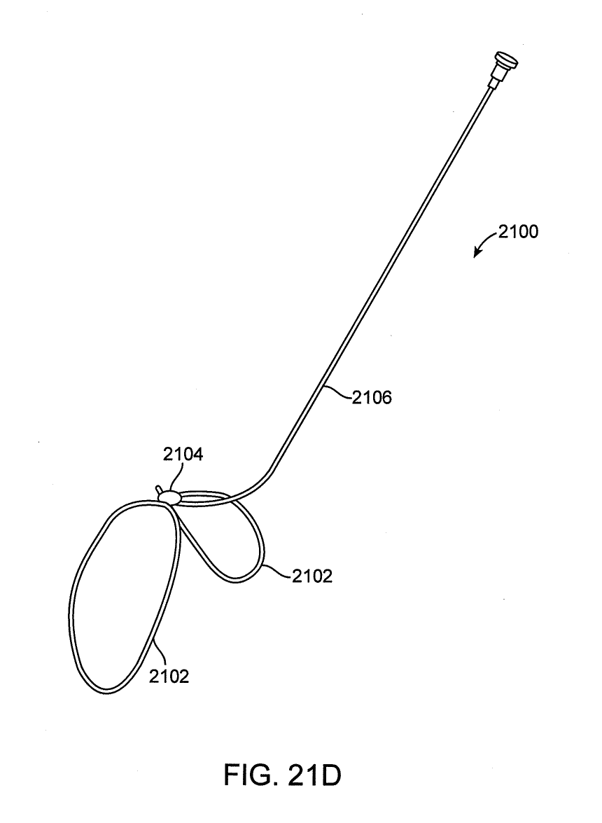

FIGS. 21A-21D shows one embodiment of a figure eight configuration for the electromagnetic treatment delivery device. As shown, the electromagnetic treatment delivery device 2100 has an applicator having a plurality of coils (multi-coil applicator) 2102. In some embodiments, the plurality of coils are conductive and non-concentric.

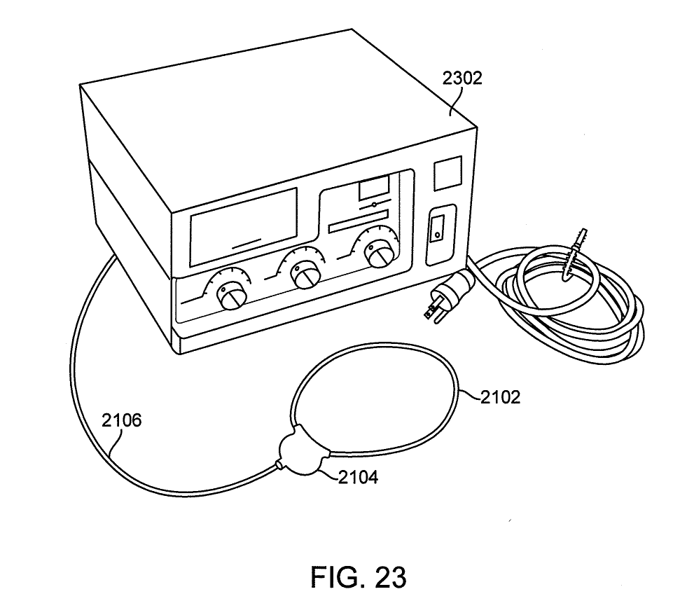

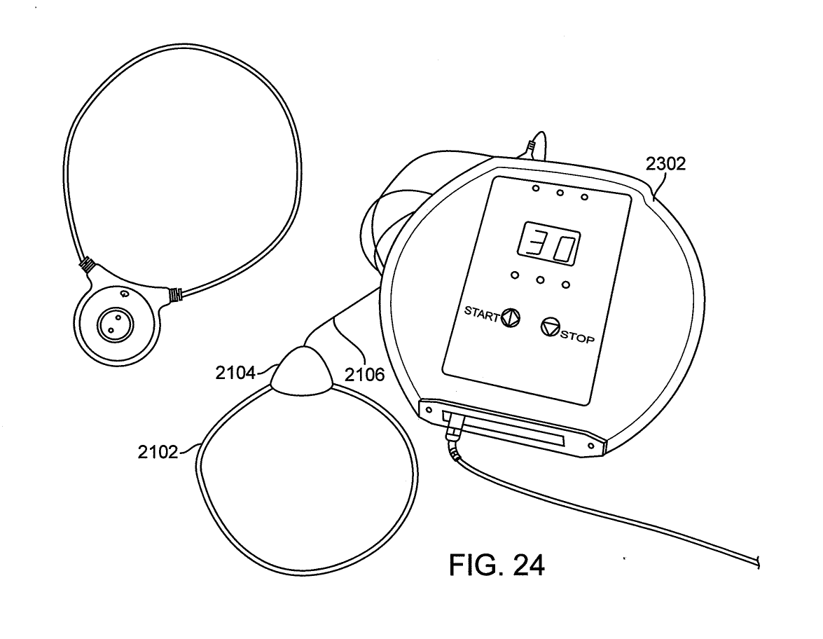

The coils 2102 of the applicator are attached to a connecting member 2104. In some embodiments, the connecting member 2104 includes tuning circuitry and components to calibrate the signal or waveform supplied to the multi-coil applicator. In some embodiments, the tuning circuit may be connected to the applicator and include a capacitor or capacitors. In some variations, the tuning circuit calibrates the frequency of a carrier signal supplied to the multi-coil applicator. In some cases, the carrier signal is tuned to 27.120 MHz. Additionally, the connecting member 2104 may be connected to a power source or a signal generator by means of a connector 2106. The connector 2106 may be connected to a signal generator/control circuit such as a SofPulse or Roma device provided by Ivivi Technologies. FIGS. 23 and 24 show the SofPulse and Roma devices 2302. Connector 2106 connects the multi-coil applicator to the signal generator 2302. FIG. 21B shows the figure eight configuration worn on a user's head. As shown, the two coils 2102 may be placed on opposing hemispheres of the user's head. Alternatively, the coils may be situated on the user's head in any suitable manner to provide treatment to multiple areas while at the same time avoiding obstruction of other medical machinery. For example, the generating coils 2102 may be placed to minimize interference with bandages. FIGS. 21C and 21D provide additional views of figure eight design for an electromagnetic treatment delivery device according to some embodiments.

In other embodiments, the applicator may include more than two coils. The applicator may comprise, for example, three coils in a clover design. In some embodiments, the plurality of coils is connected to each other by a connecting member. For example, connecting member 2104 may be used to connect multiple coils together. The connecting member 2104 may additionally connect the multi-coil applicator to a lead that connects to a power source and/or electromagnetic signal generator.

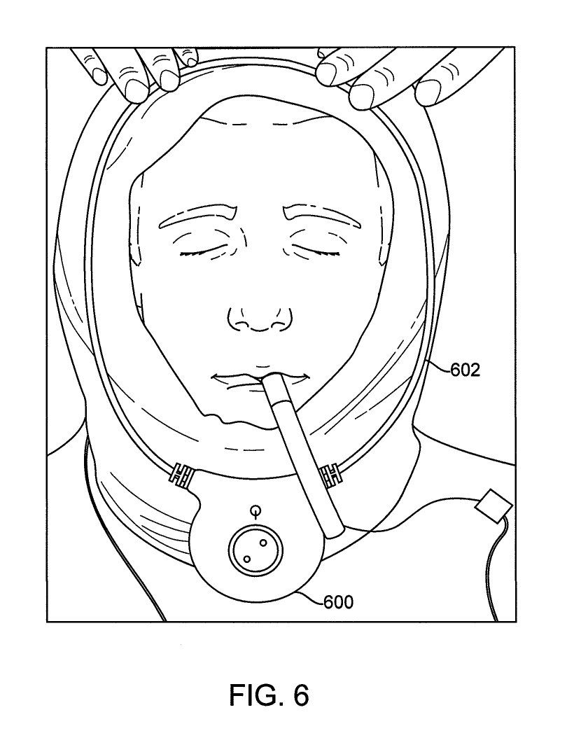

FIGS. 6, 7 and 8 show alternative embodiments where a PEMF treatment device is configured to accommodate a bandaged patient suffering from TBI. In FIG. 6, the device 600 is configured such that the generating member/applicator 602 has a sufficient diameter to encircle an anterior region of the patient's head.

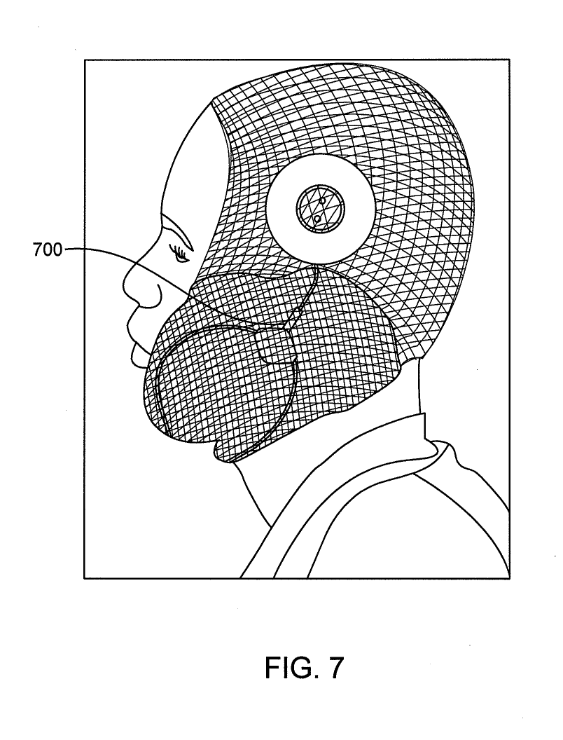

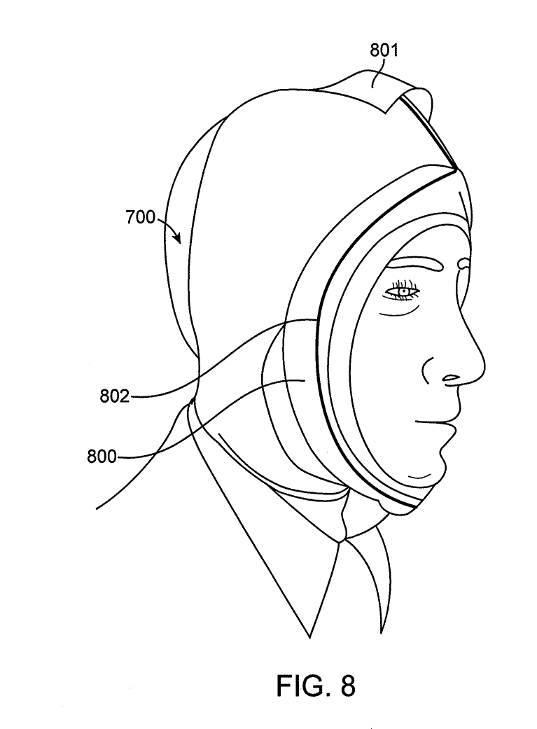

Alternatively, FIGS. 7 and 8 show embodiments that incorporate treatment devices 700 and 800 with a body support article such as a bandage or a dressing. In FIG. 7, the treatment device 700 is positioned inside the bandage such that the EMF signals are directed at the patient's neck and chin region. In FIG. 8, the treatment device 800 includes a generating member/applicator 802 that encircles the anterior portion of the patient's head and a control circuit/signal generator positioned in a top region of the bandage. In some embodiments, the bandage EMF article is disposable after use.

In some embodiments, the devices may include a sensor configured to monitor a patient's condition for changes. For example, a device may include a sensor that collects data on the patient's intracranial pressure. Based on the amount of intracranial pressure, the device may automatically turn on for treatment once threshold pressure levels are reached. Similarly, the device may turn off automatically if pressure levels return to normal. Additionally, a device providing treatment may modify and adjust treatment parameters based on the feedback from sensors. For example, a device may change treatment parameters if the sensor registers an increase in intracranial pressure. Moreover, in some variations, medical staff may be notified of changes to treatment parameters where the delivery device can communicate with another device such as computer, smart phone, printer, or other medical equipment/device.

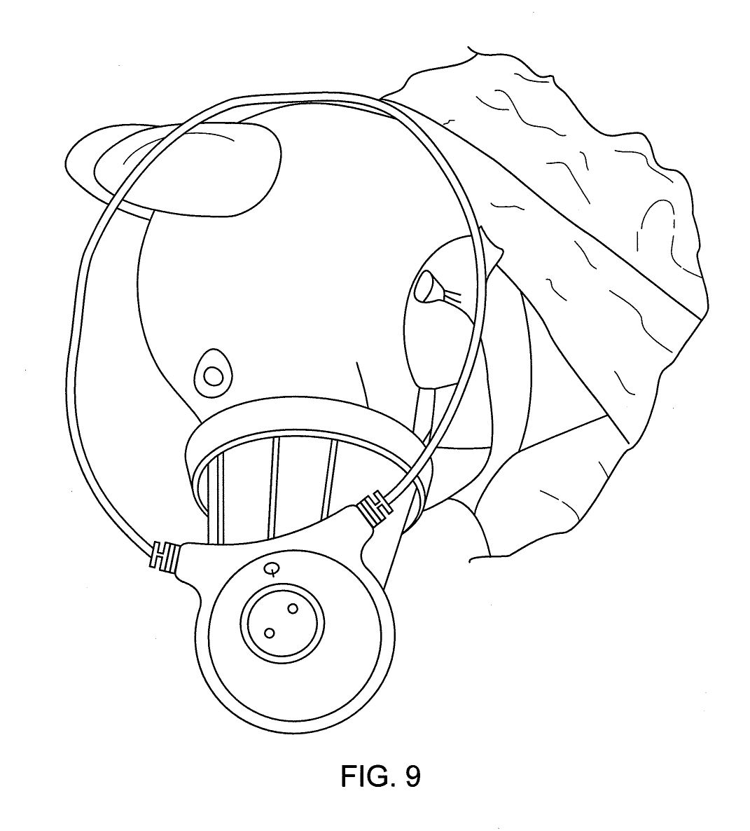

In some embodiments, treatment devices can be configured for use with non-human patient such as a canine as shown in FIG. 9.

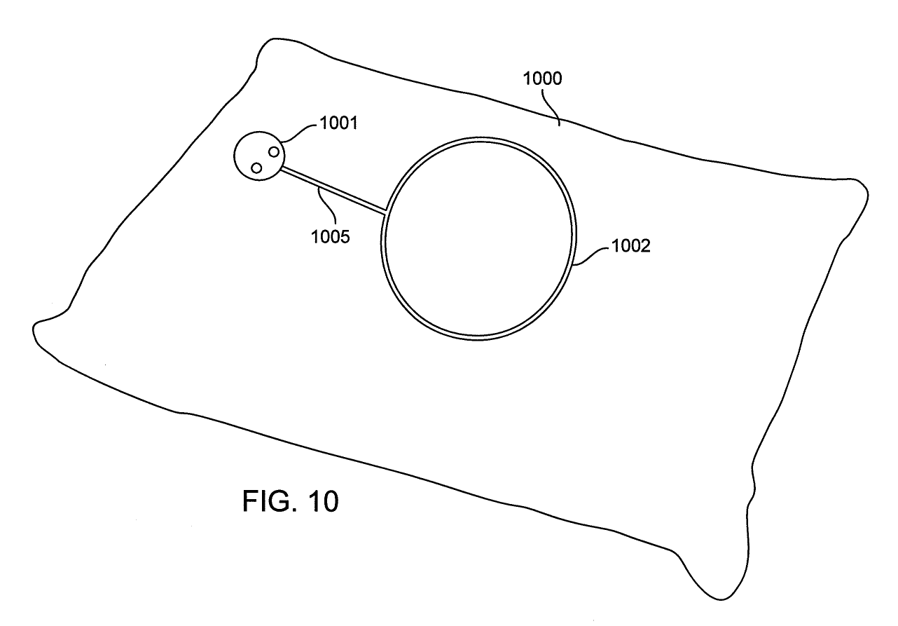

In further embodiments, the treatment methods and devices described can be incorporated into body support articles such as furniture. For example, in some circumstances, such as severe head trauma patients, use of treatment devices in bandages may not be possible or suitable. In such cases, treatment devices may be incorporated into furniture such as bedding to provide treatment with minimal interference with the patient's body and/or other ongoing treatments. For example, FIG. 10 provides an example of a treatment device 1000 incorporated into a pillow cover. In this embodiment, the treatment device 1000 includes a control circuit/signal generator 1001, a connecting member 1005, and a generating member/applicator 1002. This embodiment allows for minimal contact to the patient's head, while allowing medical staff to access the control circuit/signal generator without moving or touching the patient's head. Moreover, such embodiments reduce the amount of wiring near the patient's head which may interfere with other concurrent treatments. Additionally, if necessary, the EMF device can be removed easily and quickly in case of an emergency without disrupting the patient's other treatments. As can be appreciated, any variety of body support articles other than those described can be used in conjunction with a device to provide EMF treatment. For example, a treatment device can be incorporated into a chair, bed sheet, blanket, head board, etc.

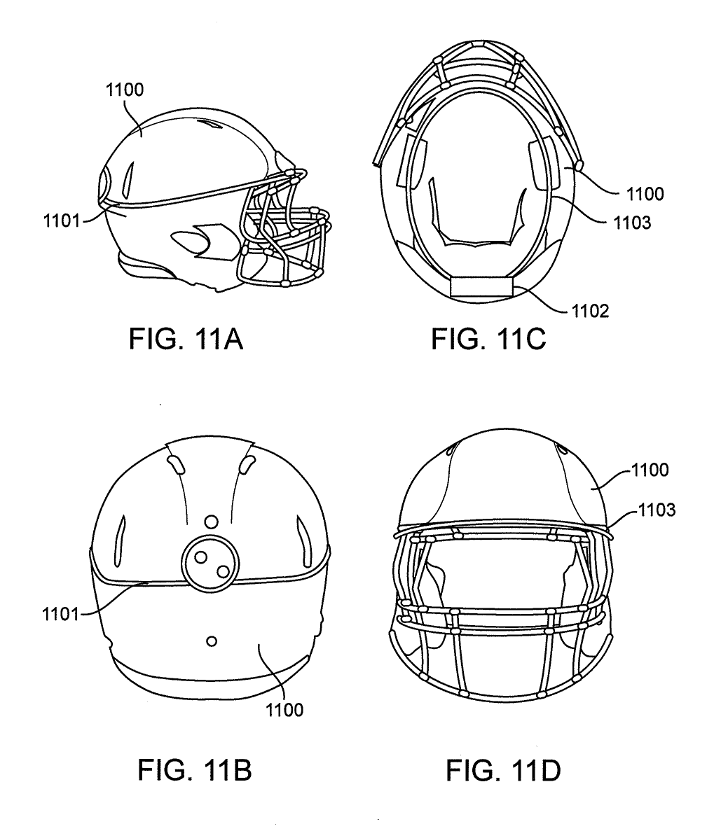

In addition, in some variations, the treatment devices and methods described can be incorporated into headgear such as a helmet or headphones to provide immediate treatment following an injury event. As shown in FIGS. 11A-11D, a treatment device 1101 can be incorporated into protective headgear 1100. The treatment device 1101 includes a control circuit/signal generator 1102 and a generating member/applicator 1103. In some embodiments, the treatment device 1101 encircles the helmet region in proximity to the cranium. In other embodiments, the treatment device, generating member/applicator, and control circuit/signal generator can be placed in any number of configurations or orientations to provide treatment from the headgear. The treatment device may be disposed within the helmet such that the treatment device is not visible on the inside or outside surfaces of the helmet. In other embodiments, the treatment device may be placed such that it is removable or detachable from a surface of the helmet. In further embodiments, a portion of the device, such as the on/off button of a control circuit/signal generator is accessible via a surface of the helmet where the remaining portions of the device are not.

In further variations, the position of the generating member(s)/applicator(s) and signal generator may be adjustable such that multiple areas of the brain may be treated at different times. For example, a subject learning new motor skills associated with skiing may need treatment in a target brain location different from a subject learning how to operate a helicopter. Advantageously, in such cases, the same headgear may be used where the position of the delivery device can be adjusted in the headgear to accommodate treatment access to different brain locations.

Additionally, the treatment device may further include remote control operability where treatment staff can modify the treatment parameters while the subject is engaged in the activity. For example, a subject engaged in learning skills for playing a football may require different cognitive abilities depending on the position the subject plays on the field. Treatment staff can provide adjustments to treatment parameters via remote control based on the cognitive processes needed.

Additionally, the treatment device may further include a sensor that can trigger the activation of the treatment device once an injurious event occurs. For example, a sensor (e.g. accelerometer) may register the force and speed of an impact and determine whether a concussion is likely to occur. In some embodiments, the sensor can provide force and speed readings to a processor in the treatment device that can automatically activate the treatment device once threshold parameters are met. Once activated, the treatment device may employ a pre-programmed EMF treatment to mitigate inflammation and swelling that is about to occur from the impact. Moreover, in some embodiments, the treatment device may alert others to the situation by providing for lights on the back of the helmet that blink or turn on to indicate the device is active. Furthermore, the device may transmit the sensor data or active status to another device such as computer, smart phone, printer, or other medical equipment/device. In such embodiments, the device may communicate through infrared or near UV signals, so as to require a specific receiver, thus concealing the activation from others in the area, such as combatants, for example. Such a device could use infrared or near UV signals, so as to require a specific receiver, thus concealing the activation from others in the area, such as combatants, for example. The sensor can be located within the signal generator on the helmet or separate from the signal generator. Depending on the space constraints of the headgear, the sensor may be placed in any number of locations suitable for gathering sufficient data to operate.

In further embodiments, device can use information from a sensor such as an accelerometer to determine the type of impact or injury experienced by a subject. The device can also apply an appropriate treatment based on the sensed information. For example, TBI or other cerebral trauma can occur from different impact forces arising from different types of triggering events. In the context of sports or accidents, a physical impact usually creates an acceleration and deceleration injury. A football player running at full sprint may contact an object or another player and experience an abrupt decelerating force on the brain or head. During rapid deceleration, a subject's brain may keep moving from inertia and impact the skull causing stress and damage to brain tissue. In such cases, a sensor can register the type of impact/force experienced by the subject and activate the device to begin an appropriate treatment for the type of injury likely to arise from that impact/force. Alternatively, head injuries can arise from other impact forces such as those experienced in combat situations. For example, military personnel may experience a head injury from a shockwave arising from a blast or explosion. In some variations, the described devices will determine the type of force causing the injury and will provide treatment appropriate for the type of injury experienced (e.g. blast wave or physical impact).

In some embodiments, the sensor may sense or measure pressure forces arising from impact, shockwaves, blast wave, or any other event that may cause neurological or physiological injury. As can be appreciated, the sensor may measure or sense or monitor any impact parameter. For example, as described above, the sensor may measure a parameter such as the impact force experienced by the user while wearing a helmet or other headgear having the sensor. Alternatively, the sensor may measure an environmental parameter such as temperature of the environment on, in, or near the sensor, or pressure and/or force exerted upon the helmet.

The sensor may be configured to sense the force of trauma or impact on the helmet or the force experienced by the user. For example, the sensor may be configured or placed on the helmet to measure the trauma force absorbed by the outer surface of the helmet. In such cases, the impact force has not been absorbed by the helmet's protective structure (e.g. padding) and the initial impact force may not be the actual impact force experienced by the user. In other cases, the sensor may be placed inside the helmet or within padding to measure the reduced impact force that is closer to the actual force experienced by the user. The reduced force may be a function of the remaining impact force experienced inside the helmet after some of the initial force has been absorbed by the helmet structure. In other embodiments, the sensor may be configured to calculate or apply an algorithm to determine the impact force experienced by the user. For example, the sensor may take into account that the helmet generally reduces initial impact forces by a certain proportion. In such cases where the initial impact force is reduced by 70%, the user would experience 30% of the original impact force inside the helmet due to the protective structure of the helmet. The sensor may be configured to activate electromagnetic field therapy only when the impact force experienced by the user exceeds a certain threshold value. The threshold value may be pre-determined. In other embodiments, the sensor may activate electromagnetic field therapy based on the measurements of the initial impact force.

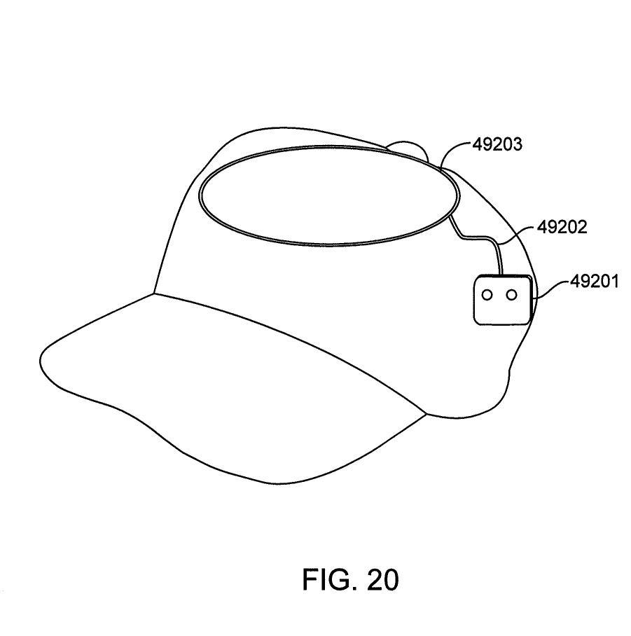

FIG. 20 shows an alternative embodiment where the delivery device is incorporated into a hat where the delivery device has generating member/applicator 49203, connecting member 49202, and signal generator 49201. Such embodiments can provide the cognitive treatment without interfering with the subject's ability to conduct activities.

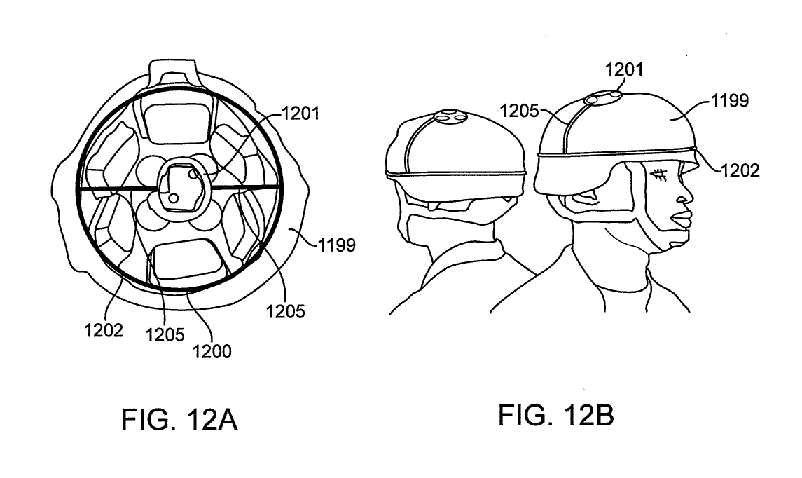

FIGS. 12A-12B provide for an alternative military headgear embodiment with an EMF treatment device. Generally, military headgear contains additional padding, which may require configuration adjustments. For example, the device 1200 can be placed within the helmet 1199 such that generating member/applicator 1202 encircles the cranium of the wearer but does not interfere with helmet padding. Similarly, the control circuit/signal generator 1201 can be disposed at the top portion of the helmet such that the on/off button can be accessed from a surface of the helmet without interfering with the helmet's effectiveness. Connecting members 1205 connect the generating member/applicator 1202 to control circuit/signal generator 1201. In further embodiments, the treatment device further includes a sensor as described above that can trigger the activation of the device once threshold parameters are met. In some embodiments, the electromagnetic delivery system can be placed near or attached to a structure of the helmet such as a shell or padding. The electromagnetic delivery system can also be incorporated into the helmet to allow for permanent or removable placement.

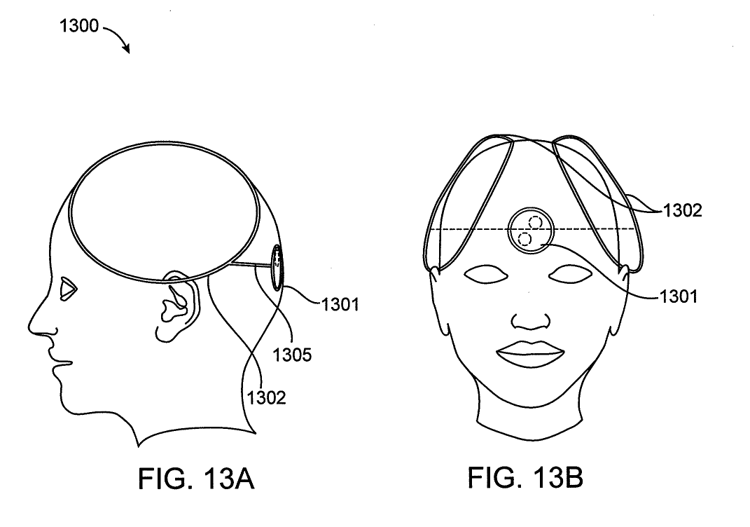

FIG. 13A-13B provide additional configurations of treatment devices 1300 where a generating member/applicator or members 1302 are placed on lateral cerebellar hemispheres and control circuit/signal generator(s) 1301 may be placed anywhere along with cranium (e.g. anterior or posterior). In some embodiments, the configurations as shown can be configured as a standard helmet insert that is removable and can be used with different types of headgear, e.g. helmets for football, motorcycle, bike, etc.

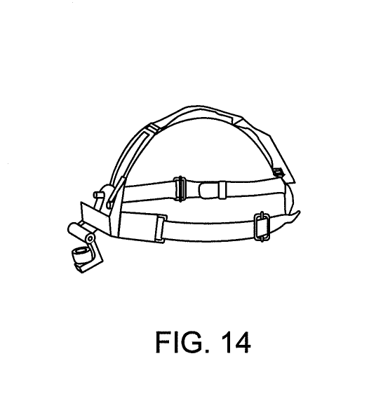

FIG. 14 shows an adjustable insert that may be used with a treatment device that can be attached and detached from headgear. In some embodiments, the insert provides support for a delivery device where the delivery device is secured in position on the helmet by the insert. The insert may include a removable securing mechanism such as Velcro that attaches to corresponding Velcro on an inner surface of the helmet. In some variations, the delivery device may be placed between the insert and the helmet such that the insert attaches the delivery device to the inner surface of the helmet.

In further variations, the adjustable insert may include a conducting material that can serve as a generating member/applicator for the signal generator. In some embodiments, an electrical wire is placed in the adjustable insert such that when the insert is placed in the headgear, it can be connected to a signal generator to provide treatment to the wearer.

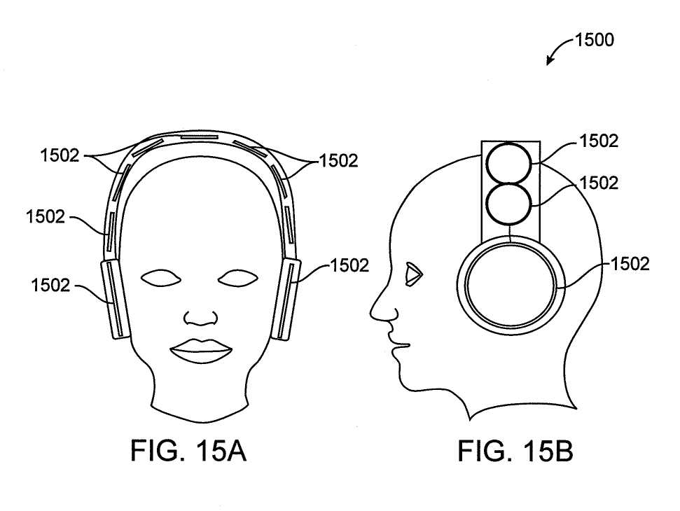

FIGS. 15A-15B provide for an alternative embodiment where the treatment device includes multiple generating members/applicators placed in an article of headgear. Headgear 1500 includes multiple generating members/applicators 1502 disposed throughout the article. In some embodiments, the control circuit/signal generator is located within the headgear. In other embodiments, the control circuit/signal generator may be located outside of the headgear and connected to the generating members/applicators by a connecting member or members.

In further embodiments, an electromagnetic field may be delivered by way of a conductive ink. In some variations, a conductive ink is applied to a material that will be placed in close proximity to a target location of the subject. For example, the conductive ink may be sprayed over a surface of an elastic headband. The conductive ink may be sprayed over the entire area of the headband or only over certain portions. The headband may be then connected to a signal generator to provide an electromagnetic field through the conductive ink on the headband to a subject. In other variations, the conductive ink is applied to a helmet or hat such that a signal generator can provide treatment through the conductive ink to the subject wearing the helmet/hat. In further embodiments, the electromagnetic field may be delivered by way of a flexible printed circuit board (PCB).

In some embodiments, the electromagnetic treatment (field or signal) is delivered by a low frequency device. In such cases, the carrier signal may have a frequency that is not in the radio frequency range. In some embodiments, the electromagnetic treatment is delivered by a signal with a frequency outside of about 3 kHz to about 300 GHz. In some embodiments, the electromagnetic treatment delivery device has a carrier signal with a frequency from about 3 MHz or lower. In other embodiments, the electromagnetic treatment delivery device has carrier signal with a frequency between about 3 MHz and about 1 Hz. In such cases, the burst width of the carrier signal may be increased. Burst widths may include 1 msec to 10 minutes. In other cases, the burst repetition may be about 1 Hz to about 0.001 Hz.

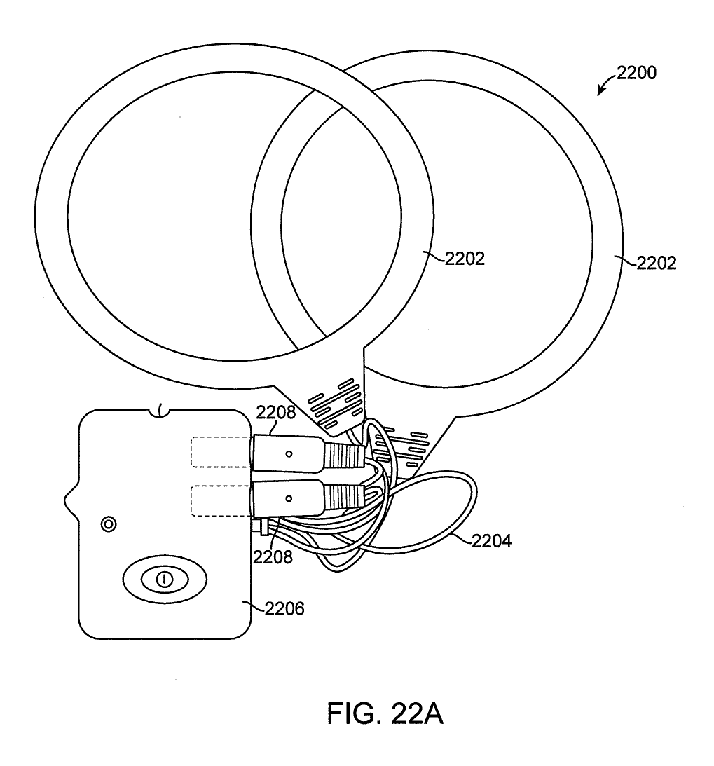

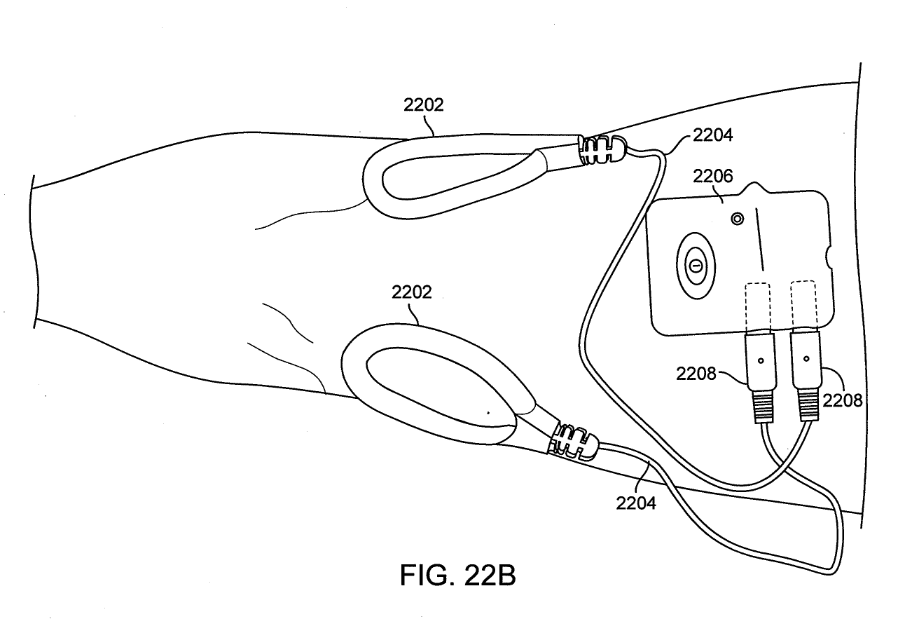

FIGS. 22A-22B provides an example of one embodiment of a low frequency device. The device 2200 has a plurality of generating members/applicators 2202 attached physically and electronically by connecting members 2204. The connecting members 2204 provide connection between the generating members/applicators 2202 and a control circuit (or signal generator) 2206. The connecting members 2204 may be removably attached to the control circuit or signal generator 2206 by any means such as a friction fit mechanism 2208. The applicator or generating members/applicators 2202 may be made out of magnetic wire, Litz wire, or a lightweight conformable wire. Additionally, any suitable configuration may be used. As shown in FIGS. 22A-22B, the generating members/applicators form loops that can be placed on either side of the knee. In other embodiments, the generating members/applicators may be connected to form a figure eight design as described previously.

In further embodiments, the low frequency electromagnetic device may be useable without a tuning circuit. As described above, in some embodiments, the electromagnetic delivery device includes a tuning circuit to calibrate the delivered electromagnetic signal to a particular set of parameters including waveform frequency. For low frequency electromagnetic delivery devices, a tuning circuit may be omitted.

In further embodiments, the low frequency electromagnetic device utilizes a low amount of power such as below about 5 watts.

Another aspect of the invention provides for systems, methods, and devices for a treatment session with a combination of electromagnetic field treatment and cognitive training. In some embodiments, an electromagnetic field delivery device, such as those described above, delivers an electromagnetic field to a patient's target brain region while the patient also undergoes cognitive training. In some cases, the cognitive training is targeted at the same brain region receiving the electromagnetic field treatment. In other embodiments, the cognitive training is targeted at a different region from the electromagnetic field; however, the cognitive function may be the same one treated.

Some systems may include a processor configured to activate the electromagnetic field treatment and cognitive training exercises. The cognitive training may be timed to occur while a level of a physiological effect in the brain region caused by the electromagnetic field is above a predetermined level. Additionally, repeated cycles of electromagnetic field treatment and cognitive training may be provided to increase the effectiveness of the treatment. In some cases, the cognitive training starts immediately after termination of the electromagnetic field treatment. In other cases, the cognitive training occurs before or during the delivery of therapeutic electromagnetic field to the target region. The cognitive training may continue for about 10-1000 seconds or longer and/or repetitive, as the training requires.

The electromagnetic field treatment and/or cognitive training may be directed towards any single or multiple neurological regions such as brain regions associated with, for example, Alzheimer's disease, dementia, mild cognitive impairment, memory loss, aging, ADHD, Parkinson's disease, depression, addiction, substance abuse, schizophrenia, bipolar disorder, memory enhancement, intelligence enhancement, concentration enhancement, well-being or mood enhancement, self-esteem enhancement, language capabilities, verbal skills, vocabulary skills, articulation skills, alertness, focus, relaxation, perceptual skills, thinking, analytical skills, executive functions, sleep enhancement, motor skills, coordination skills, spots skills, musical skills, interpersonal skills, social skills and affective skills.

Additionally, any one or more of the brain regions stimulated by the delivered electromagnetic field or cognitive training may be, for example, a left prefrontal region, frontal lobe, cingulated gyms, nispheres, temporal lobe, a parietal lobe, occipital lobe, amygdale ion, cerebellum, hippocampus, anthreonal, Peabody, plaques, tangles, brain stem, dula, corpus collasum, subcortical region, cortex, gyrus, white matter, or gray matter.

In some embodiments, the cognitive training may be directed towards tasks specifically designed to improve memory retention, face-name associations, object-location associations, performance on a prospective memory task, reality orientation, implementation of various cognitively stimulating tasks as questioning/memorizing current events, solving simple computerized crossword puzzles and labyrinth etc. The cognitive training may be visual stimulation, audio stimulation, olfactory stimulation, tactile stimulation, spatial stimulation.

Additionally, the cognitive training may be selected to train the same or different region as treated by the electromagnetic field. Examples of areas of the brain (and associated cognitive training) that can be included for treatment are described in U.S. patent application Ser. No. 12/285,416 filed on Jan. 24, 2011, which is herein incorporated by reference in its entirety.

The stimulation provided by PEMF may be sub-threshold, meaning that it does not typically result in firing (either inhibitory or excitatory) of action potentials. The very low energy PEMF signals described herein may result in substantial and measurable cognitive effects. The PEMF maybe configured, as described herein, to target a molecular pathway implicated in cognition, such as the NO pathway.

In some embodiments, the brain areas targeted may be directed toward those affected by Alzheimer's Disease. Examples of cognitive training exercises correlated with affected brain regions include: syntax and grammar tasks for the Broca area; comprehension of lexical meaning and categorization tasks for the Wernicke area; action naming, object naming and spatial naming (of shapes, colors, and letters) tasks for both the R-dlPFC and the LdlPFC areas; and spatial attention (for shapes and letters) tasks for both R-pSAC and L-pSAC areas.

Some embodiments provide a system for neurological treatment comprising: (a) a PEMF delivery device (b) a cognitive training exercise targeted for at least one brain region; (c) a processor configured to execute a treatment session where the treatment session comprises treating at least one brain region with PEMF and coordinating cognitive training in conjunction with the PEMF. The system of the invention may be used, for example, in the treatment of any form of dementia or other age related diseases, in the treatment of any form of neurological conditions, or in the treatment of any form of psychiatric conditions.

The delivery of PEMF to a target brain region may cause a predetermined physiological effect. The physiological effect may have an initial level that decays in time after termination of the PEMF treatment. The physiological effect may or may not be an effect that is quantifiable by anyone or more of fMRI, EEG, PET, SPECT, cognitive measures, EMG and MEP.

In other embodiments, the treatment session may include i=1 to M, where M is a number of brain regions, for j=1 to N(i), where N(i) is a number of times a first brain region i is to be treated by PEMF, and N(i) is at least 2, (a) activating an electromagnetic field delivery device for a predetermined amount of time; and (b) providing cognitive training to deliver cognitive training to a second brain region i, the cognitive training being started at a predetermined time relative to the activation period of the PEMF treatment.

In some embodiments, the system includes a cognition training device. The cognition training device may include a display screen and a subject input device such as a keyboard. The display screen is disposed so as to be conveniently viewed by a subject, and the input device is positioned so as to be conveniently accessible to the subject. In some cases, a processor controls the cognitive training device. The processor may include a memory for storing data relating to training protocols, data relating to the subject, such as MRI images, as well as storing data relating to training sessions. The processor may be configured to register the electromagnetic field delivery device. The processor may execute one or more predetermined treatment protocols, collect a subject's response to cognitive training delivered during a training session, store the collected data in the memory, and analyze the data.

A treatment session can involve treating one or more brain regions, or the entire brain. In some cases, PEMF treatment is delivered to cause a physiological effect. Once a physiological effect is elicited, the cognitive training device is then activated to deliver cognition training to the brain region during the duration of the physiological effect. In some embodiments, the cognition training is started while the level of the physiological effect is above a predetermined fraction of the initial level. In other embodiments, the cognitive training is provided before, after, or during the PEMF treatment (which may or may not be correlated with a detected or detectable physiological effect. This cycle of PEMF delivery with cognitive training may be repeated several times, to ensure the effectiveness of the treatment session. The next episode of PEMF treatment may be initiated sufficiently soon after the previous episode of PEMF, to ensure that the effect does not decay below a predetermined fraction of the initial level during the treatment regime. In further embodiments, the delivered electromagnetic field does not cause excitatory or inhibitory synaptic response or event.

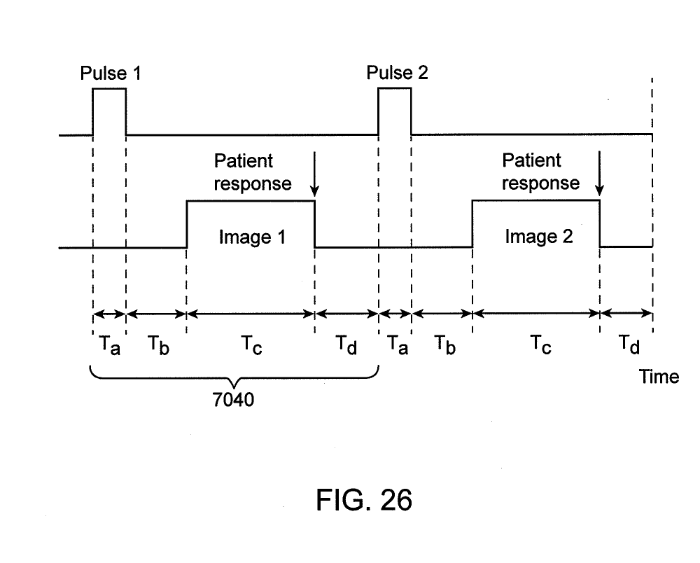

FIG. 26 shows an exemplary treatment protocol for a first given brain region. The protocol commences with a first cycle 7040 consisting of PEMF treatment during a time period Tₐ, which may be for example, 0.1-10 sec, preferably 1-4 sec. followed by a first interlude of duration T_{b} (of duration, for example, between 0 to 10 sec) which is then followed by cognitive training during a time period T_{c} (of duration, for example, between 5 to 300 seconds, preferably 10-60 sec), and a second interlude of duration T_{d} (between 0 to 10 sec). The time interval T_{b}+T_{c}+T_{d} may be selected to be sufficiently short that the effect is above a predetermined fraction of the initial level that was present at the termination of the PEMF delivery.

Another aspect of the invention provides for systems, methods, and devices for diagnosing and treating various neurological conditions and/or for modifying (e.g. enhance) at least one of cognitive, behavioral, or affective functions or skills in individuals. Some embodiments provide for a non-invasive PEMF device configured to modify a cognitive function for a target or identified brain area. The PEMF device may be any suitable PEMF device including any of those described above and shown in FIGS. 2-16, 20, and 21-24.

In some embodiments, the method for improving or enhancing a cognitive function may include the steps of: (i) non-invasively providing a PEMF signal to a target region of a patient's head and therefore brain; and (ii) improving or enhancing a cognitive feature associated or correlated with the target region. The method may also include providing training or conditioning related to the target region of the patient's head (e.g., associated with the function of the target region) during and/or immediately after the PEMF application. In some embodiments, the PEMF signal provided is in the ISM band.

Other embodiments provide for PEMF systems for enhancing particular cognitive, behavioral, or affective functions (or skills) in brain-related cognitive functions in normal individuals. In some cases, a determination of “normal” cognitive function is based on a comparison of the individual's structural or functional or cognitive functioning with corresponding statistical health or brain diseases norms or with statistical norms for cognitively enhanced functions. Further embodiments provide for neurological diagnostic computational systems and methodology for diagnosing an individual with a brain-related disease or diseases, along with a specification of the individual's functional, structural, or cognitive abnormalities. In alternative embodiments, the invention provides diagnostic computational systems and methodology for identifying cognitive function or functions, which may be further enhanced in an individual.

Other embodiments provide for PEMF devices, methods, and systems for treating one or more brain regions (or other neurological regions) to enhance or improve corresponding cognitive functions, while continuously monitoring and adjusting the treatment parameters for a given individual or a disease or a particular cognitive enhancement function, based on a comparison of pre- and post-stimulation diagnostic measurements of the relevant brain function, structure, and corresponding cognitive functions.

Further embodiments provide for PEMF devices, methods, and systems for locating a diseased brain regions or regions and delivering therapeutic PEMF stimulation to improve cognitive performance in a particular skill or skills in normal individuals. The PEMF stimulation may be combined with convergent cognitive stimulation of the same brain regions, and/or with in-vivo regenerative or neuronal implantation of neuroplasticity methodologies that can initiate a regeneration, replacement, or growth of the same brain regions, to maximize the potential therapeutic or neuroplasticity effect, or with any pharmaceutical agent or material which may facilitate the neuroplasticity or regenerative or enhancement of cognitive functions associated with the same brain region or regions being treated.

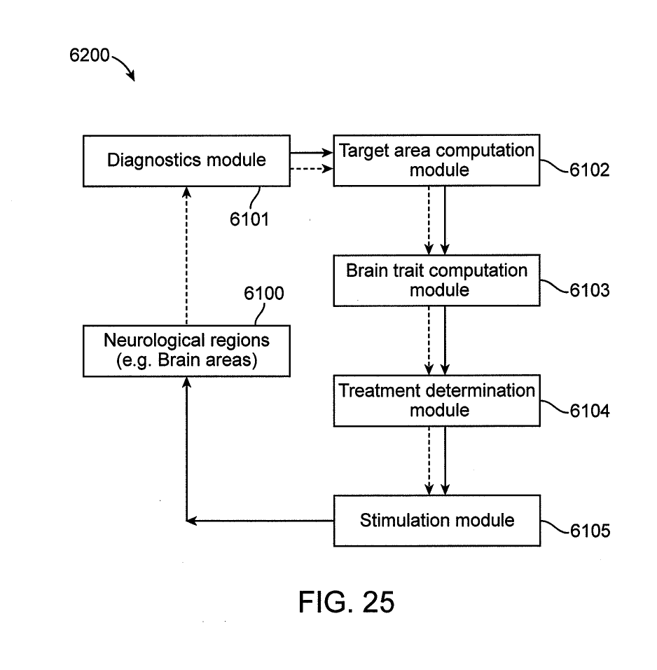

Reference is made to FIG. 25, which illustrates neurological regions 6100 that are pathological functional or structural brain features, or cognitive performance features in an individual. These regions may be brain regions that are associated or correlated with a specific brain-related disease. In some embodiments, a diagnostic step or module 6101 may be used to detect and/or measure functional activation or structural maps, or corresponding cognitive performance in an individual for a particular task (or tasks) or during a resting period. The diagnostics module 6101 can communicate this information to a target area computation module 6102. The target area computation module 6102 can identify neurological or brain regions in an individual whose structure, function, or cognitive functions deviate or differ from corresponding statistically-established health norms, or from corresponding statistical norms for cognitively enhanced performance in a particular task.

In some embodiments, the diagnostics module 6101 compares an individual's neuroimaging data with statistically established health norms to determine whether the individual has normal cognitive function. This neuroimaging data can be obtained through the use of various magnetic resonance imagining (MRI), functional magnetic resonance imagining (fMRI), positron emission tomography (PET), single photon emission computerized tomography (SPECT), electroencephalography (EEG) and event related potentials (ERP) techniques, among many others.

In further embodiments, information regarding the individual's cognitive performance may be considered. For example, measurements of cognitive performance of an individual in a wide range of possible cognitive or behavioral tests, which may include but are not limited to: response times, accuracy, measures of attention, memory, learning, executive function, language, intelligence, personality measures, mood, and self-esteem, among others may be considered by the diagnostics module 6101.

In some embodiments, the individual's neuroimaging data and cognitive performance measurements are analyzed in the diagnostics module 6101. Based on the analysis of the diagnostics module 6101, an appropriate PEMF treatment can be determined for the individual for enhancing or improving cognition.

In other embodiments, the target area computation module 6102 of system 200 is configured to identify a particular functional or structural brain region, or corresponding cognitive characteristics, that are different in a given normal individual from their corresponding attributes in statistical standard of excellence or enhanced performance in a particular cognitive skill or function associated with a particular brain region. This may be accomplished, in some embodiments, by assessing an individual's cognitive functions or abilities and comparing those individual functions or abilities with statistically established health norms in terms of functional activation patterns, structure, or corresponding cognitive performance levels. If there is a difference or deviation between the individual's abilities and the statistical norm, PEMF treatment may be provided to enhance or improve the individual's cognitive function. Alternatively, where the individual's cognitive function does not deviate from the statistical norm, PEMF treatment may still be provided to enhance cognitive (or behavioral) performance beyond an initial level. In some embodiments, the comparison between the individual's ability and an established norm may be carried out by any procedure known in the art. For example, comparison of the individual's functional activation patterns, brain structure or cognitive performance to statistically-established norms of functional, structural, or cognitive performance in individuals who exhibit excellent cognitive performance in a particular task or skill can rely on a statistical contrast of the individual's pixel by pixel, or region by region, functional and structural or cognitive performance values with the corresponding values of a normally-distributed healthy control group or population.

FIG. 25 also shows that the target area computation module 6102 can communicate with a brain trait computation module 6103. The brain trait computation module 6103 can receive information that is output from the target area computation module 6102. The target area computation module may output identified statistically-deviant or cognitively-enhanced brain regions in a given individual for analysis in the brain trait computation module 6103. The brain trait computation module 6103 may, in some embodiments, determine whether or not any of these identified brain regions statistically fits within known structural, functional, or cognitive pathophysiology of a particular brain-related disease. Alternatively, the brain trait computation module 6103 may determine whether or not any of these identified brain regions statistically fits within established norms for enhanced or excellent cognitive or behavioral performance (in a particular task or skill or skills). For example, as described in U.S. patent application Ser. No. 12/285,416 filed on Oct. 3, 2008 (herein incorporated by reference in its entirety), in the case of Autism Spectrum Disorder (ASD), statistically-established norms indicate that autistic children or individuals exhibit an abnormal deficient activation (as well as structurally decreased size) of the left hemisphere's (LH) typical Broca's and Wernicke's language regions, while abnormally hyperactivating (or structurally enlarged) contralateral (RH) Broca's and Wernicke's regions. In such cases, a target computation module 6102 may identify an abnormal hypoactivation of the LH's Broca's and Wernicke's language regions (with or without an accompanying hyperactivation of the contralateral RH's Broca's and Wernicke's regions). The target computation module 6102 may then output the regions to the brain trait computation module 6103.

Alternatively, in the case of Alzheimer's disease (or any other memory loss that is due to aging, dementia or mild cognitive impairment (MCI)), memory impairment is often correlated with decreased structure and function of the hippocampus and other medial temporal structures, as well as decreased connectivity between frontal and posterior brain regions and facial recognition regions, or structural, functional, or cognitive impairment of the cerebellum (associated with impaired motor coordination and semantic memory or verbal capability loss), or impairment of mood and executive functioning regions (such as the left prefrontal region and cingulate gyrus and frontal lobe). In cases where the target area computation module 6102 identifies such abnormally-decreased structural or functional values of these brain structures, these brain regions are output to the brain trait computation module 6103, to determine whether or not any of these identified brain regions statistically fits within known structural, functional, or cognitive pathophysiology of Alzheimer's, MCI, dementia, or age-related memory loss, or other aging illnesses. If the identified regions of interest or cognitive performance levels match the brain disease, or match the neural functional, structural, or cognitive levels of a sub-cognitively enhanced performance in a particular task or tasks, the treatment determination module 6104 may compute the individual-based brain and cognitive treatment parameters needed to stimulate the identified brain regions to improve the functional, structural or cognitive disease indices, or to enhance performance in a particular task or tasks.

In some embodiments, the target area of computation module 6102 can output identified cognitively enhanced brain regions in a given individual for analysis in the brain trait computation module 6103 for analysis on whether any of the identified regions deviates from the established norms for enhanced or excellent cognitive or behavioral performance (in a particular task or skill or skills). Thus, for instance, in the case of a normal individual whose cognitive functions may be found to be different from those for enhanced cognitive functions, PEMF treatment may be provided to identify sub-enhanced brain regions to improve cognitive function. In some embodiments, treatment determination module 6104 may compute precise individual-based brain and cognitive PEMF stimulation parameters for improving cognitive function(s) geared towards enhancing performance in a particular task or tasks.

Some embodiments provide for methods, systems, and devices for computing parameters for PEMF treatment to optimize neuroplasticity. In some cases, optimization of neuroplasticity may be employed for treating Alzheimer's memory loss, dementia, memory loss diseases, or memory enhancement diseases. PEMF treatment may be provided to the hippocampus or other temporal lobe regions or frontal or prefrontal regions or cingulate gyrus in any possible combination. In some embodiments, PEMF treatment is provided with or synchronized with memory enhancement or encoding or retrieval or recall or recognition or mnemonic or perceptual or auditory or semantic memory enhancement cognitive training or stimulation methodologies, to obtain the optimal neuroplasticity potential changes related to memory improvement.

Referring back to FIG. 25, stimulation module 6105 receives input from the treatment determination module 6104. In some embodiments, the stimulation module 6105 receives PEMF neuro-cognitive stimulation parameters from the treatment determination module 6104. In some embodiments, feedback may be also combined with the stimulation module 6105 and feedback may include a post-stimulation measurement carried out by the diagnostics module 6101. In some embodiments, the feedback allows for ongoing monitoring and adjusting the individual-based brain and corresponding cognitive stimulation parameters continuously. In some embodiments, the system described monitors potential improvement in functional, structural, or corresponding cognitive stimulation in an individual following the administration of treatment and may adjust treatment based on the improvement. In other embodiments, the feedback system will monitor and adjust treatment until a certain cognitive enhancement threshold has been reached or exceeded.

In some embodiments, the treatment determination module 6104 is configured to determine the appropriate PEMF treatment parameters for brain, cognitive, and neuro-cognitive stimulation for an individual with a neurological condition, and/or the appropriate location (brain region) to apply PEMF. Alternatively, the treatment determination module 6104 may determine the appropriate therapeutic electromagnetic field treatment parameters for brain, cognitive and neuro-cognitive stimulation parameters for a normal individual to enhance a particular cognitive function. For example, a treatment determination module 6104 may indicate a treatment parameter of a pulse-modulated radio frequency signal at 27.12 MHz. In other embodiments, the electromagnetic treatment signal may have at a 2 msec burst repeating at about 2 bursts/sec.

In some embodiments, the stimulation module 6105 provides for a PEMF cognition treatment separately or together with cognitive training. For example, an electromagnetic treatment with a signal that is 27.12 MHz carrier pulse-modulated can be coupled with a computerized, auditory, or visual presentation of a Beck-based “positive thinking,” or change in self-construct cognitive stimulation or training paradigm, which may be juxtaposed together in any possible order and with any temporal separation between their onset, termination time, and length of stimulation. Similarly, any PEMF treatment can be coupled with short term memory cognitive exercises or attention allocation exercises. PEMF treatments could also be paired with cognitive stimulation or training geared towards diminishing the likelihood of occurrence of false-perceptions (e.g., through enhanced perceptual training such as enhancing perceptual cues in perceptual illusion paradigms or other perceptual paradigms or, alternatively, through enhancing accurate perception training or through cognitive stimulation or training in enhancing attention or attentional allocation capabilities, or increasing psychophysical judgment capabilities). In other embodiments, individuals who have been characterized as possessing functional, structural, or cognitive abnormalities that are characteristic of autism may be treated with PEMF stimulation of the LH's Broca's and Wernicke's regions with cognitive or behavioral stimulation geared towards enhancing language development, articulation, naming, pointing, or joint attention skills, among others.

In further embodiments, PEMF treatment can be provided to the Amygdala or fusiform gyrus (which have been shown to be hyperactivated in ASD individuals during facial recognition and social cognition tasks, or during non-social communication paradigms or even at resting conditions) during resting conditions or during the conductance of non-social cognition tasks—which may be coupled with focused social cognition stimulation exercises (before or after the PEMF stimulation during the resting state or non-social communication tasks).

In some embodiments, the PEMF treatment may be combined with a cognitive exercise or training. The PEMF and cognitive training may be conducted at the same time or separately. The cognitive treatment may be of single or multiple presentation of various sensory modality stimulation such as visual, auditory, and tactile, for example, with various response modalities being used in any possible combination, including but not limited to a keypress response, vocal, written, tactile, or visually guided response with or without a response feedback element (e.g., which provides a feedback as to the accuracy of the subject's response or performance at different time points, or with regards to various segments of the task or tasks at hand).

Each of the components of FIG. 25 can function independently or separately, or in any possible combination with each other. In some embodiments, the diagnostics module 6101 can translate functional or structural neuroimaging data into statistically valid individual functional activation patterns and statistically valid individual structural maps. The diagnostics module 6101 may also be configured to compare an individual's cognitive performance data with statistically established health norms.

In order to enhance various cognitive functions or skills the corresponding brain regions can be targeted for PEMF treatment, e.g., hippocampus or temporal lobe or cingulated gyrus for memory or learning enhancement, frontal or prefrontal cortex for executive functions, concentration, learning, intelligence; motor cortex or cerebellum for motor functions and coordination, visual cortex for enhancing visual functions, inhibitive amygdale for fear and anxiety reduction with or without left frontal and prefrontal stimulation; enhancement of self-esteem or mood or well-being-stimulation of left prefrontal or frontal, or stimulation of the right prefrontal gyrus.

For Alzheimer's, target regions for treatment may include abnormally deficient activation of left frontal, left prefrontal, Broca's, Wernicke's, hippocampus and related regions, anterior cingulated, and also motor, medial temporal gyms, anthreonal gyrus, cerebellum, and a decline in functional connectivity measures between some or all of these regions. Structural abnormalities may also exist as a decrease in these structures' volume or connecting fibers between these neuronal regions.

For autism spectrum disorder, targets regions for treatment may include reversed functional activation of right hemisphere RH instead of left hemisphere LH language regions activation patterns in ASD children (and adults) relative to normal matched controls (e.g., hypoactivation of LH's Broca's, Wernicke's regions but hyperactivation of these contralateral regions in the RH in the ASD relative to matched controls). For “Theory of Mind” social cognition ASD deficits, functional hypoactivation of the Amygdala, fusiform gyrus, and dysfunction of inter-hemispheric connectivity measures may occur. Additionally, a generalized RH dysfunction in the ASD individuals relative to controls which may manifest as a generalized RH hyperactivation in Theory of Mind paradigms, at resting conditions or in language paradigms, may occur.

As discussed above, some embodiments provide for a system with both PEMF treatment and cognitive training. The system may include a processor (e.g., computer) and a PEMF delivery device. The computer may supply cognitive stimuli during the PEMF treatment. In some cases, the treatment and cognitive training is conducted under the supervision of an operator. In other cases, the patient or user may undergo treatment and training at home. The progress of cognitive training (and the training itself) may be conducted on a mobile device that communicates progress to a medical professional. The patient may undergo treatment in any position-upright, sitting, reclined, etc.

In some embodiments, neuroimaging may be used to identify changes in the treatment region over time. For example, MRI images may be used to observe the progress of the PEMF treatment and cognitive training in a particular brain structure. The images may provide the caregiver or offsite personnel input on the best stimulation locations and training regime for the individual. In some embodiments, this may include determining the exact coordinates of the location to be stimulated on the patient and the optimal cognitive training to use in conjunction with the stimulation. Other embodiments provide PEMF treatment to brain region/s in order to enhance a particular cognitive function or functions or skill/s.

In some embodiments, a feedback loop measures the patient's functional or structural or neuroplasticity or neurophsyiological state prior to single or multiple sessions of electromagnetic and/or cognitive stimulation and also following such single or multiple treatment sessions. This feedback loop may adjust the corresponding PEMF stimulation and cognitive training.

In some embodiments, a script is used to enhance or improve cognition. The script can indicate the cognitive training to be applied, the time delay between the applied electromagnetic field and the cognitive exercise. The script can also include graded responses to patient feedback allowing determination of patient's progress, responses being tagged with scores for determination of patient's progress. Scripted stimuli to the patient at appropriate intervals before, after, or during PEMF treatment. Patient feedback in the forms of answers or responses to the cognitive stimuli may be collected in real-time.

For any of the described embodiments, PEMF treatment parameters may all be dynamically changed or adjusted based on the post-treatment results.

### EXAMPLE 1

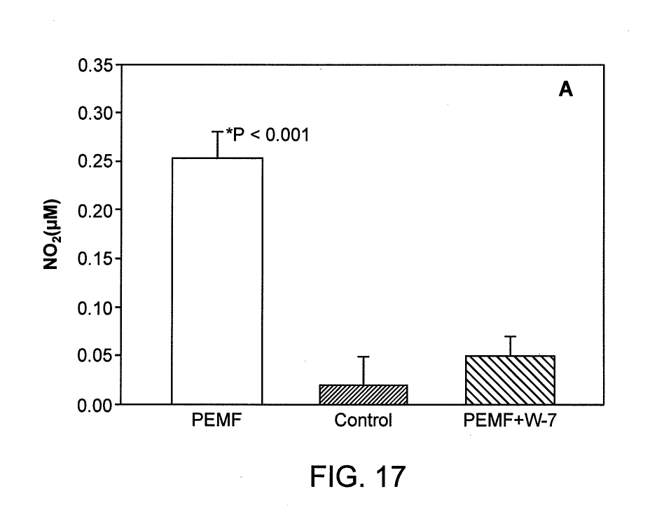

In this example experiments, designed to assess the EMF effect on NO release, were performed on a dopaminergic cell line (MN9D) in culture. Cells were plated at 100,000 cells/35 mm dish in Dulbecco's Modified Eagle's medium (DMEM) containing 10% fetal calf serum and allowed to stabilize for 24 hours. Thereafter, serum was withdrawn and cells allowed to stabilize for 6 hours at 37° C. These cultures were placed at room temperature for 15 min to create a repeatable stress which caused cytosolic Ca²⁺ to rise, thereby activating CaM. Cells were then treated for 15 min with a non-thermal RF signal configured according to the teachings of this application, which consisted of a 27.12 MHz carrier pulse-modulated with a burst duration of 3 msec at 2 bursts/sec. In situ signal amplitude was 0.05 G which induced a mean electric field of approximately 18 V/m. The results in FIG. 17 show the EMF signal increased NO production by several-fold, and that this was inhibited by N-(6-Aminohexyl)-5-chloro-1-naphthalenesulfonamide hydrochloride (W-7), a CaM antagonist. These results demonstrate that an EMF signal configured according to the present invention can modulate CaM-dependent NO signaling.

### EXAMPLE 2

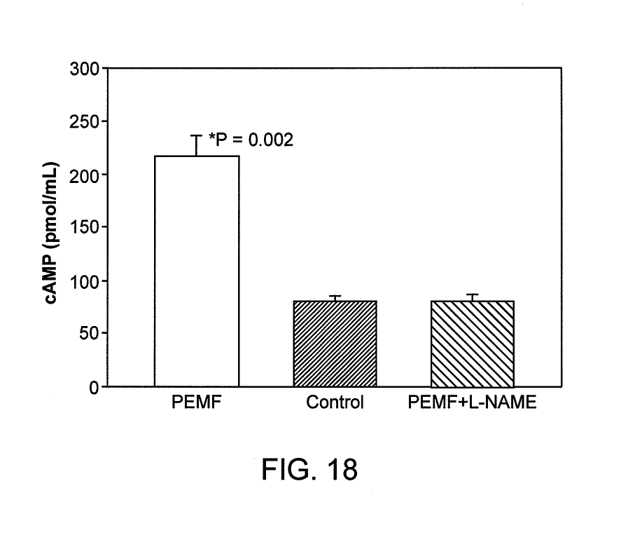

In this example experiments, designed to assess the EMF effect on cAMP release, were performed on a dopaminergic cell line (MN9D) in culture. Cells were plated at 100,000 cells/35 mm dish in Dulbecco's Modified Eagle's medium (DMEM) containing 10% fetal calf serum and allowed to stabilize for 24 hours. Thereafter, for the cAMP signaling experiments, serum was withdrawn and cells allowed to stabilize for 6 hours at 37° C. Cells were then treated for 15 min with a non-thermal RF signal configured according to the teachings of this application, which consisted of a 27.12 MHz carrier pulse-modulated with a burst duration of 3 msec at 2 bursts/sec. In situ signal amplitude was 0.05 G which induced a mean electric field of approximately 18 V/m. The results in FIG. 18 show the EMF signal increased cAMP production approximately 2-fold, and that this was inhibited by L-nitrosoarginine methyl ester (L-NAME), a cNOS inhibitor. These results demonstrate that an EMF signal configured according to the present invention can modulate the CaM dependent signaling pathway related to neuronal cell differentiation (plasticity).

### EXAMPLE 3

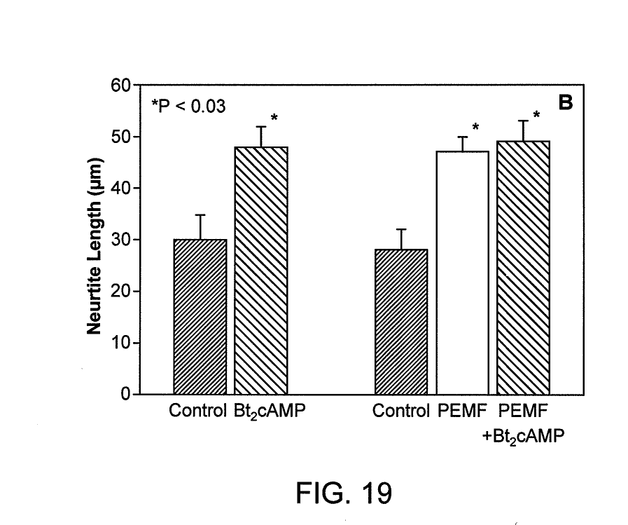

In this example experiments, designed to assess the EMF effect on neurite outgrowth (differentiation), were performed on a dopaminergic cell line (MN9D) in culture. Cells were plated with or without fetal calf serum and 1 mM dibutyryl cyclic adenosine monophosphate (Bt2cAMP). At 1 day, immature cultures were divided into two groups and treated with a non-thermal RF signal configured according to the teachings of this application, which consisted of a 27.12 MHz carrier pulse-modulated with a burst duration of 3 msec at 2 bursts/sec. In situ signal amplitude was 0.05 G which induced a mean electric field of approximately 18 V/m. EMF treatment was 30 minutes a day for three days. Cultures assigned to control groups were exposed to the same conditions in the absence of EMF signals. After three days of treatment, cells were fixed and photographed for subsequent analysis with ImageJ. Measurements of neurite length, cell numbers, and number of cells with and without processes were quantified in 4 consecutive fields under phase optics at 100× magnification. Process lengths less than 10 μm were excluded. Data were analyzed with the Student's t-test. P<0.05 was considered significant. The results in FIG. 19 show EMF produced a 43% additional increase in neurite length (P=0.03), compared to the control group. Effects of this EMF signal on differentiation were also compared with those of exogenous cAMP a known inducer of neurite outgrowth. It was found that addition of 1 mM Bt2cAMP significantly increased neurite length by 41% (P=0.001). However PEMF treatment in the presence of cAMP did not further increase neurite length, suggesting that this was achieved through a common mechanism that reached its maximum effect with this concentration of the cyclic nucleotide. These results illustrate that an EMF signal configured according to this invention can modulate neuronal differentiation which, in turn, modulates cognitive processes, as well as neuronal repair.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A protective helmet apparatus for delivering electromagnetic treatment comprising:
a helmet shell having an opening adapted to receive the head of a user;
at least one layer of padding within the helmet shell configured to provide comfort and reduce impact forces on the head of the user;
an electromagnetic treatment device at least partially within the helmet shell, the electromagnetic treatment device comprising:
an applicator configured to deliver a therapeutic electromagnetic field to the user's head; and
a control circuit controlling a generator configured to provide an electromagnetic signal to the applicator to induce the therapeutic electromagnetic field; and
a sensor coupled to the helmet, the sensor configured to detect an impact parameter and to activate the electromagnetic treatment device when the impact parameter exceeds a predetermined threshold.

2. The apparatus of claim 1, comprising a plurality of applicators positioned to apply an electromagnetic field sequentially or simultaneously to specific cerebral regions.

3. The apparatus of claim 1, wherein the electromagnetic signal comprises a carrier signal having a frequency in a range of about 0.01 Hz to about 10,000 MHz and a burst duration from about 0.01 to about 1000 msec and a burst repetition rate of about 0.1 to 100 Hz.

4. The apparatus of claim 1, wherein the electromagnetic signal comprises a repetitive pulse burst, wherein each pulse may be symmetrical or asymmetrical, wherein each pulse has a duration of about 10⁻⁸ sec to 10⁻¹ sec and a burst duration from about 0.01 to about 1000 msec and a burst repetition rate of about 0.1 to 100 Hz.

5. The apparatus of claim 1, wherein the sensor is an accelerometer.

6. The apparatus of claim 1, wherein the sensor is a pressure sensor.

7. The apparatus of claim 1, wherein the electromagnetic treatment device is configured to apply a pre-programmed treatment protocol.

8. The apparatus of claim 1, further comprising an alert means for indicating that the electromagnetic treatment device is active.

9. The apparatus of claim 1, wherein the sensor measures an impact force experienced by the user.

10. The apparatus of claim 1, wherein the sensor measures a shockwave force experienced by the user.

11. The apparatus of claim 1, wherein the electromagnetic treatment device is removable from the helmet.

12. The apparatus of claim 1, wherein the applicator is configured to contact the user's scalp.

13. The apparatus of claim 1, wherein the electromagnetic treatment device comprises a replaceable or rechargeable power source.

14. The apparatus of claim 1 further comprising a remote control element configured to operate the electromagnetic treatment device.

15. The apparatus of claim 1, wherein the applicator comprises pliable and conformable coils having a generally circular shape.

16. The apparatus of claim 1, wherein the applicator has a diameter between about 6 inches to about 8 inches.

17. The apparatus of claim 1, wherein the applicator is adjustable.

18. The apparatus of claim 1, wherein the applicator comprises a collapsible wire having a retracted and extended position.

19. The apparatus of claim 1, wherein the applicator is removably attached to the helmet with a fastening mechanism.

20. The apparatus of claim 1, wherein the applicator comprises conductive ink.

21. The apparatus of claim 1 further comprising a connecting member between the applicator and the control circuit.

22. The apparatus of claim 21, wherein the connecting member comprises a pliable material adapted to allow the applicator and the control circuit to move relative to each other.

23. The apparatus of claim 1 further comprising a processor configured to collect and record user information while the apparatus is worn.

24. The apparatus of claim 1, wherein the electromagnetic device is configured to emit a pulse-modulated radio frequency signal at 27.12 MHz at a 2 msec burst repeating at about 2 bursts/sec.

25. The apparatus of claim 1, wherein the electromagnetic signal comprises a carrier signal below 1 MHz.

26. The apparatus of claim 1, wherein the electromagnetic signal generated by the control circuit and generator has a carrier frequency within the ISM band.

27. The apparatus of claim 1, wherein the electromagnetic signal generated by the control circuit and generator has a carrier frequency of approximately 27.12 MHz.

28. The apparatus of claim 1, wherein the electromagnetic signal is configured to modulate the production of cytokines and growth factors produced by living cells.

29. The apparatus of claim 27, wherein the cytokines and growth factor cells are produced by neuronal cells.

30. The apparatus of claim 27, wherein the cytokines and growth factors cells are modulated in response to cognitive or neurological conditions or injury.

31. The apparatus of claim 1, wherein the electromagnetic signal is configured to modulate signaling.

32. The apparatus of claim 1, wherein the electromagnetic signal is configured to enhance a release of NO in response to cognitive or neurological conditions or injury.

33. The apparatus of claim 1, wherein the electromagnetic signal is applied in conjunction with imaging, non-imaging and electrophysiological monitoring, such as MRI, fMRI, SPECT, PET, EEG, EMG, etc.

34. The apparatus of claim 1, wherein the electromagnetic signal is controlled by a program which depends upon the information received from imaging, non-imaging and electrophysiological monitoring.

35. The apparatus of claim 1, wherein the electromagnetic signal is applied to a single or a plurality of applicators placed to target specific cerebral areas in a sequence determined by the therapeutic goals and requirements as monitored by imaging, non-imaging and electrophysiological measures.

36. An electromagnetic treatment delivery device comprising:
a multi-coil applicator configured to apply a therapeutic electromagnetic field to multiple locations on a user's head, wherein the multi-coil applicator comprises a plurality of non-concentric conductive coils;
a control circuit configured to control a generator , wherein the generator is coupled to the multi-coil applicator and configured to provide a pulse-modulated radio frequency signal to the multi-coil applicator to induce the therapeutic electromagnetic field.

37. The device of claim 36, wherein the control circuit is configured to direct the multi-coil applicator to target a single or a plurality of cerebral regions in a sequence.

38. The device of claim 37, wherein the control circuit is configured to direct the multi-coil applicator to target a single or a plurality of cerebral regions in a sequence determined by imaging, non-imaging and electrophysiological monitoring.

39. The device of claim 36, further comprising a connecting member connecting the plurality of conductive coils to each other and to the generator.

40. The device of claim 36 further comprising an article of headwear configured to be worn by a user, wherein the multi-coil applicator is incorporated into the headwear.

41. The device of claim 36, wherein the multi-coil applicator forms a figure eight pattern.

42. The device of claim 36, wherein the multi-coil applicator comprises pliable and conformable coils having generally circular shapes.

43. The device of claim 36, wherein at least two coils of the multi-coil applicator each have a diameter between about 2 inches to about 8 inches.

44. The device of claim 36, wherein the multi-coil applicator is configured to generate an electric field on at least two hemispheres of the user's head.

45. The device of claim 36, wherein the device is incorporated into a bandage or dressing.

46. The device of claim 36 further comprising a sensor configured to monitor a user parameter.

47. The device of claim 46, wherein the user parameter is intracranial pressure.

48. The device of claim 36, wherein the control circuit is configured to control the device to deliver a pre-programmed treatment protocol.
